(19) United States
(12) Patent Application Publication
(10) Pub. No.: US 2015/0225758 A1
(43) Pub. Date: Aug. 13, 2015

(54) GENETICALLY MODIFIED CELLS AND METHODS FOR MAKING ACTIVATED SUGAR-NUCLEOTIDES

(71) Applicant: UNIVERSITY OF GEORGIA RESEARCH FOUNDATION, INC., Athens, GA (US)

(72) Inventors: Maor Bar-Peled, Athens, GA (US); Ting Yang, Changchun (CN)

(73) Assignee: UNIVERSITY OF GEORGIA RESEARCH FOUNDATION, INC., Athens, GA (US)

(21) Appl. No.: 14/356,291

(22) PCT Filed: Nov. 8, 2012
(86) PCT No.: PCT/US2012/064055

§ 371 Date: May 5, 2014

Related U.S. Application Data
(60) Provisional application No. 61/557,161, filed on Nov. 8, 2011.

(51) Int. Cl. C12P 19/30; C07H 19/10; C07H 19/207; G01N 33/58

(52) U.S. Cl. CPC C12P 19/305; G01N 33/58; C07H 19/10; C07H 19/207

(57) ABSTRACT

This disclosure generally relates to genetically engineered cells and methods of making and using such genetically engineered cells. Generally, the genetically engineered cells exhibit an increase in synthesis of an activated sugar-nucleotide compared to a wild type control. In some embodiments, the activated sugar-nucleotide produced by the genetically engineered cell is an activated sugar-nucleotide that is not natively synthesized by wild type, un-engineered cell. In some embodiments, the activated sugar-nucleotide is an activated uridine diphosphate sugar nucleotide. In other embodiments, the activated sugar-nucleotide is an activated cysteine monophosphate sugar nucleotide. In still other embodiments, the activated sugar-nucleotide is an activated guanosine diphosphate sugar nucleotide. In some embodiments, the activated sugar-nucleotide includes an isotopic label.

### CROSS-REFERENCE TO RELATED APPLICATION

This application claims priority to U.S. Provisional Patent Application Ser. No. 61/557,161, filed Nov. 8, 2011, which is incorporated herein by reference.

### GOVERNMENT FUNDING

The present invention was made with government support under Grant No. IOS-0453664, awarded by the NSF and Grant No. DE-AC05-00OR22725, awarded by the DOE. The Government has certain rights in this invention.

### SUMMARY

In one aspect, this disclosure describes a genetically engineered cell that exhibits an increase in synthesis of an activated sugar-nucleotide compared to a wild type control. In some embodiments, the activated sugar-nucleotide produced by the genetically engineered cell is an activated sugar-nucleotide that is not natively synthesized by wild type, un-engineered cell.

In some embodiments, the activated sugar-nucleotide is an activated uridine diphosphate sugar nucleotide. In other embodiments, the activated sugar-nucleotide is an activated cysteine monophosphate sugar nucleotide. In still other embodiments, the activated sugar-nucleotide is an activated guanosine diphosphate sugar nucleotide.

In some embodiments, the activated sugar-nucleotide includes an isotopic label.

In some embodiments, the genetically engineered cell is a genetically engineered prokaryotic cell. In other embodiments, the genetically engineered cell is a eukaryotic cell.

In another aspect, this disclosure describes a method for making an activated sugar-nucleotide. Generally, the method includes providing a genetically engineered cell exhibiting increased phosphorylation of a monosaccharide sugar at the 1 position to produce a monosaccharide-1-phosphate compared to a wild-type bacterial cell; and culturing the cell in the presence of the monosaccharide to yield the activated sugar-nucleotide.

In some embodiments, the monosaccharide sugar is galactose and the activated sugar-nucleotide is UDP-galactose. In other embodiments, the monosaccharide sugar is galacturonic acid and the activated sugar-nucleotide is UDP-galacturonic acid. In still other embodiments, the monosaccharide is glucose and the activated sugar-nucleotide is UDP-rhamnose.

In some embodiments, the method further includes isolating the activated sugar-nucleotide.

In another aspect, this disclosure describes an alternative method for making an activated sugar-nucleotide. Generally, this method includes providing a genetically engineered cell exhibiting increased production of an activated sugar-nucleotide compared to a wild-type cell; and culturing the cell under conditions suitable for production of the activated sugar-nucleotide.

In some embodiments, the activated sugar-nucleotide is a UDP sugar-nucleotide, a CMP sugar-nucleotide, or a GDP sugar-nucleotide.

In some embodiments, the method further includes isolating the activated sugar-nucleotide. In some embodiments, the method includes culturing the genetically engineered cell in a medium that includes a labeled carbon source. In alternative embodiments, the method includes culturing the genetically engineered cell in a medium that includes a labeled nitrogen source.

In another aspect, this disclosure describes an isotopically labeled activated sugar-nucleotide. In some embodiments, the isotopic label includes ²H, 13C, ¹⁴C, ¹⁵N, ¹⁷O, ¹⁸O, ³²P, or ³³P.

In yet another aspect, this disclosure describes a method that generally includes performing an assay using the isotopically labeled activated sugar-nucleotide as summarized immediately above. In some embodiments, the assay can include nuclear magnetic resonance and the isotopically labeled activated sugar-nucleotide is used as a standard. In another embodiment, the assay can include mass spectrometry and the isotopic labeled activated sugar-nucleotide is used as a standard. In some of these embodiments, the method can further include tracking movement of the isotopic label. In other embodiments, the method can further include imaging the isotopic label.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. A model for NDP-sugar synthesis in an engineered cell. Sugar(s) or sugar precursor(s) can be either taken up by the bacterium and biotransformed to new NDP-sugar(s) or endogenous NDP-sugar can be used for biotransformation into new activated sugars. Exemplary nucleotide sugars produced by recombinant enzymes are shown in bold. The enzymes and coding regions are designated in italics. Existing pathways for the formation of nucleotide sugars by engineered E. coli, the host cell in one exemplary embodiment, are indicated by arrows with dashes: UDP-GlcNAc is formed through Frc and Gln.

FIG. 2. Analyses of UDP-GlcNAcA and UDP-XylNAc produced engineered biological by engineered E. coli using anion-exchange chromatography and ESI-MS/MS. Cell cultures (of engineered E. coli expressing UDP-GlcNAc dehydrogenase, panel A; UDP-GlcNAc dehydrogenase and UDP-XylNAc synthase, panel D; or plasmid control, panel B and E) were extracted four hours after induction. An aliquot from each sample was separated by Q15-HPLC (Gu and Bar-Peled, Plant Physiol. (2004) 136:4256-4264) and analyzed at A_{261 nm}(panels A, B, D, and E). The peaks labeled #1 (panel A) and #2 (panel D) were collected and analyzed by ESI-MS, shown in panel C and panel F, respectively. Panel A shows the formation of UDP-GlcNAcA (marked by arrow #1) in engineered cell when compared with control cell (panel B); panel D shows the formation of UDP-XylNAc (marked by arrow #2) in engineered cells when compared with control (panel E). The labeled peaks in panels A and D correspond to UDP-GlcNAcA (23.2 minutes) and UDP-XylNAc (16.0 minutes), respectively. Panels C and F show the negative ion mode MS/MS analysis of the parent [M-H]⁻ UDP-sugars and their MS/MS fragments (see details in Table 2)

FIG. 3. 2D-¹³C—HSQC NMR spectra of the HPLC-purified UDP-[¹³C]GlcNAcA. 600 MHz 2D-¹³C—¹H-NMR spectra of UDP-GlcNAcA purified from engineered E. coli cells (harboring UDP-GlcNAc dehydrogenase) grown in the presence of uniformly labeled [¹³C]glucose. At this concentration, the unlabeled uracil 5 and 6 C—H signals are not detectable.

FIG. 4. 2D-¹⁵N-HMBC NMR spectra of the HPLC-purified [¹⁵N]UDP-GlcNAcA. 600 MHz 2D-¹⁵N—¹H-NMR spectra of UDP-GlcNAcA purified from engineered E. coli cells (harboring UDP-GlcNAc dehydrogenase) grown for four hours in the presence of [¹⁵N]NH₄Cl. The atoms highlighted in red are ¹⁵N-labeled.

FIG. 5. 2D-¹³C—HSQC NMR spectra of the HPLC-purified UDP-[¹³C]XylNAc. 600 MHz 2D-¹³C—¹H-NMR spectra of UDP-XylNAc purified from engineered E. coli cells (harboring UDP-GlcNAc dehydrogenase and UDP-XylNAc synthase) grown for four hours in the presence of uniformly labeled [¹³C]glucose. At this concentration, the unlabeled uracil 5 and 6 C—H signals are not detectable.

FIG. 6. 2D-¹⁵N-HMBC NMR spectra of the HPLC-purified [¹⁵N]UDP-XylNAc. 600 MHz 2D-¹⁵N—¹H-NMR spectra of UDP-XylNAc purified from engineered E. coli cells (harboring UDP-GlcNAc dehydrogenase and UDP-XylNAc synthase) grown in the presence of [¹⁵N]NH₄Cl. The atoms highlighted in red are ¹⁵N-labeled.

FIG. 7. Characterization of UDP-Gal and UDP-GalA produced engineered biological using anion-exchange chromatography and ESI-MS/MS. E. coli (expressing GalK and Sloppy coding regions, panel A; GalAK and sloppy, panel D; or plasmid control, panel B and E) were grown for four hours in the presence of the additives as indicated and the NDP-sugars were then extracted. An aliquot from each sample was separated by Q15-HPLC (Gu and Bar-Peled, Plant Physiol (2004) 136:4256-4264) and monitored at A_{261 nm} (panels A, B, D, and E). Panel A shows the formation of UDP-Gal (arrow #3) in engineered cells supplemented with galactose (Gal) and Panel B is shows control cells supplemented with Gal. Panel D shows the formation of UDP-GalA (marked by arrow #4) in engineered cells supplemented with galacturonic acid (GalA) and Panel E is shows the control cells supplemented with GalA. The HPLC peaks in panels A and D are UDP-Gal (16.8 minutes) and UDP-GalA (23.8 minutes), respectively. Panels C and F show the MS/MS analysis performed at the negative mode of the parent UDP-sugars. The parent ions and fragmentations are listed in Table 2.

FIG. 8. Time course for engineered biological production of NDP-sugars. E. coli cells harboring UDP-GlcNAcDH (light line) or UDP-GlcNAcDH and UDP-XylNAcS (dark line) were grown and an aliquot was removed after addition of IPTG (time 0), and then at hourly intervals. The amounts of NDP-sugars (UDP-GlcNAcA (diamond) and UDP-XylNAc (circle)) produced were plotted. Each value (g/ml) is the mean of triplicate reaction, and the value varied by no more than 5%.

FIG. 9. LC/MS/MS analysis of CMP-KDO produced by E. coli expressing recombinant CMP-KDO synthase. The analysis was performed at the negative mode, the parent CMP-KDO (negative ion at m/z 543.373) gives an MS/MS fragment of m/z 322 [CMP-H]⁻¹.

FIG. 10. ¹H-NMR spectrum of UDP-GlcNAcA, produced by recombinant UDP-GlcNAc dehydrogenase. Peak eluted from Q15 column (see FIG. 2, Arrow #1) was collected, lyophilized, dissolved in D₂O and analyzed by ¹H-NMR. The NMR spectrum (2.0-8.0 ppm) covering the sugar anomeric region is shown in panel “a”. A more detailed spectrum that covers the NDP-sugar carbon ring (3.5-4.4 ppm) is shown in panel “b”. H refers to protons of the uracil ring; H′ refers to protons of the ribose ring, and H″ refers to protons of the sugar carbon ring. Unlabeled peaks are column contaminants.

FIG. 11. ¹H-NMR spectrum of UDP-XylNAc, produced by recombinant UDP-GlcNAc dehydrogenase and UDP-XylNAc synthase. Peak eluted from Q15 column (see FIG. 2, Arrow #2) was collected, lyophilized, dissolved in D₂O and analyzed by ¹H-NMR. The NMR spectrum (2.0-8.0 ppm) covering the sugar anomeric region is shown in panel “a”. A more detailed spectrum that covers the NDP-sugar carbon ring (3.7-4.4 ppm) is shown in panel “b”. H refers to protons of the uracil ring; H′ refers to protons of the ribose ring, and H″ refers to protons of the sugar carbon ring. Unlabeled peaks are column contaminants.

FIG. 12. ¹H-NMR spectrum of UDP-Gal, produced by recombinant Sloppy and GalK. Peak eluted from Q15 column (see FIG. 7, Arrow #3) was collected, lyophilized, dissolved in D₂O and analyzed by ¹H-NMR. The NMR spectrum (3.5-8.0 ppm) covering the sugar anomeric region is shown in panel “a”. A more detailed spectrum that covers the NDP-sugar carbon ring (3.7-4.4 ppm) is shown in panel “b”. H refers to protons of the uracil ring; H′ refers to protons of the ribose ring, and H″ refers to protons of the sugar carbon ring. Unlabeled peaks are column contaminants.

FIG. 13. ¹H-NMR spectrum of UDP-GalA, produced by recombinant Sloppy and GalAK. Peak eluted from Q15 column (see FIG. 7, Arrow #4) was collected, lyophilized, dissolved in D₂O and analyzed by ¹H-NMR. The NMR spectrum (3.7-8.0 ppm) covering the sugar anomeric region is shown in panel “a”. A more detailed spectrum that covers the NDP-sugar carbon ring (3.8-4.5 ppm) is shown in panel “b”. H refers to protons of the uracil ring; H′ refers to protons of the ribose ring, and H″ refers to protons of the sugar carbon ring. Unlabeled peaks are column contaminants.

FIG. 14. Metabolic conversion of [¹³C]glucose to UDP-[¹³C]GlcNAc. The UDP-GlcNAc biosynthesis pathway in E. coli. A [¹³C]Glc can be transformed into [¹³C]GlcN-1P and [¹³C]Glc can also metabolize to decorate the acetate moiety of Acetyl-CoA.

FIG. 15. Metabolic conversion of [¹³C]glucose to 1,5P-[¹³C]Rib via the pentose shunt, and the formation of UMP with orotic acid. In E. coli, [¹³C]glucose is metabolized to 5-phospho-alpha-D-[¹³C]Ribose-1-diphosphate (PRPP). The orotic acid along with PRPP are condensed and transformed to UMP and then phosphorylation to UTP.

FIG. 16. Contribution of glutamine and aspartic acid residues to the synthesis of orotic acid and the formation of the uracil ring. The moieties from glutamine are enclosed in a solid box. The nitrogen moiety labeled with ¹⁵N (comes from [¹⁵N]glutamine) is highlighted in bold. The moieties from aspartic acid are enclosed in a dotted box.

FIG. 17. Metabolic conversion of [¹³C]glucose to labeled UDP-rhamnose in E. coli engineered to over-express fungal genes involved in UDP-rhamnose synthesis. (A) and (B) show HPLC analysis of UDP-rhamnose produced by E. coli expressing recombinant Sloppy, fungal UDP-glucose-4,6-dehydratase (from Botryotinia fuckeliana), and the fungal UDP-4-keto-6-deoxyglucose-3,5-epimerase/-4-reductase (from Magnaporthe oryzae). The major NDP-sugar produced when the engineered cells were grown in regular medium is UDP-rhamnose, eluting from the column at ˜12 minutes (A). UDP-rhamnose is also the major peak when the culture medium was supplemented with [¹³C]glucose (B). (C) LC/MS/MS analysis was performed using direct injection of total NDP-sugar extract to Shimadzu spectrometer operating at the negative mode. The parent unlabeled UDP-rhamnose (negative ion at m/z 549.05) gives an MS/MS fragment of m/z 323 [UMP-H]⁻¹ (E). (D) LC/MS/MS analysis of activated sugar-nucleotide derived from culture supplemented with [¹³C]glucose, shows three molecular species of UDP-rhamnose: the unlabeled form m/z 549.05; the labeled form with m/z 555.07 where the labeled is incorporated at each of the six carbons of rhamnose; and a labeled form of m/z 560.08, where the ¹³C is incorporated at each of the five carbons of ribose and each of the six carbons of Rha. (F) MS/MS of the parent ion of the labeled UDP-Rha 560.08 gives UMP with m/z 323. (G) MS/MS of labeled [¹³C]UDP-[¹³C]Rha gives UMP with m/z 328.

### DETAILED DESCRIPTION OF EXEMPLARY EMBODIMENTS

Nucleotide sugars (NDP-sugars) are the sugar donors used for the formation of polysaccharides, glycoproteins, proteoglycans, glycolipids and for the synthesis of glycosylated secondary metabolites (Bar-Peled and O'Neill, Annu. Rev. Plant Biol. (2011) 62:127-155) and glycosylated antibiotics (Luzhetskyy et al., Curr. Top. Med. Chem. 8 (2008) 680-709). At least 70 different nucleotide sugars have been identified in bacteria and 30 activated sugars have been detected in plants. By contrast, humans and fungi are believed to synthesize 10 and up to 15 activated sugars, respectively. Although virtually all organisms can produce UDP-glucose and GDP-mannose, few organisms are capable of forming ADP-glucose, TDP-glucose, GDP-glucose or CDP-glucose. Moreover, some nucleotide sugars may be unique to a group of organisms. For example, only land plants have been shown to synthesize UDP-apiose.

Different metabolic pathways exist for the formation of nucleotide sugars. For example, photosynthetic organisms fix CO₂ and use it to generate fructose, which can then enter various metabolic pathways for the synthesis of activated sugars including, for example, GDP-Man, UDP-Glc, ADP-Glc, and UDP-GlcNAc. On the other hand, bacteria, fungi, and mammals rely on acquired carbon for making precursors that enter nucleotide sugar metabolic pathways.

We recently identified a set of operons in the Gram-negative bacterium Bacillus cereus subsp. cytotoxis NVH 391-98 that contain coding regions that encode proteins that catalyze the formation of the seldom observed sugar nucleotides UDP-2-acetamido-2-deoxy-xylose (UDP-XylNAc) and UDP-2-acetamido-2-deoxy-glucuronic acid (UDP-GlcNAcA) (Gu et al., J. Biol. Chem. (2010) 285:24825-24833). The bacterium contains other operons that may function to generate UDP-GlcA (and possibly TDP-GlcA) and UDP-GalA (and possibly TDP-GalA). Each of the operons also contains coding regions annotated as glycosyl transferases that may use these activated sugars for glycan synthesis. To study these putative glycosyltransferases we require a convenient supply of the appropriate activated sugars. We found that purified recombinant enzymes were not cost-effective for synthesizing UDP-XylNAc and UDP-GlcNAcA in amounts sufficient for such studies. Furthermore, to generate ¹⁵N-labeled or ¹³C-labeled UDP-XylNAc and UDP-GlcNAcA required the use of additional enzymes to form the appropriate isotopically labeled monosaccharides.

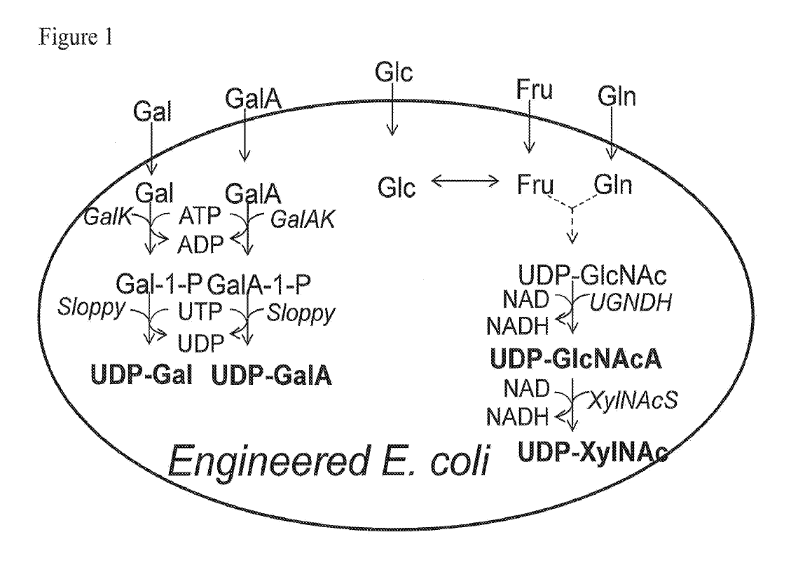

Thus, we developed an engineered biological system that uses a recombinant cell that has been modified to contain one or more polynucleotides that encode proteins that can convert a sugar or a sugar precursor into its corresponding sugar-1-phosphate and subsequently into the desired NDP-sugar. The engineered biological system can be adapted to produce a broad range of rare and common activated sugar metabolites including, for example, UDP-GlcNAcA, UDPXylNAc, UDP-Gal, and UDP-GalA (FIG. 1) and others (see, e.g., Table 1). The engineered biological system may permit one to tailor an engineered cell to, for example, convert endogenous NDP-sugars to other NDP-sugars, convert one or more fed sugar to NDP-sugars, produce isotope-labeled NDP-sugars, and/or produce short-lived NDP-sugars. The engineered biological system also can permit the biotransformation of NDP-sugar(s) to glycans such as, for example, glycolipids, polysaccharides, glycoproteins, and secondary metabolites.

### Engineering a Cell to Produce UDP-GlcNAcA and UDP-XylNAc

The description that follows frequently refers to one exemplary embodiment in which the engineered recombinant cell is E. coli. As described in more detail below, however, the genetically engineered cells and related methods described herein may involve other recombinant cells. Thus, the description referring to E. coli as an exemplary genetically engineered microbe shall not be limiting. Similarly, the following description refers to particular enzymes, coding regions that encode those enzymes, and particular source organisms for the enzymes/coding regions that are used to transform a host cell to produce the recombinant cell. As further described in more detail below, the engineered biological system is not limited to the particular enzymes, encoded by the exemplified coding regions obtained from the exemplified source organisms, but may be practiced using any suitable enzyme or combination of enzymes exhibiting the desired activity. Moreover, the enzyme may be encoded by any suitable coding region obtained from any suitable organism and operably linked to a coding region suitable for expressing the coding region in the host cell.

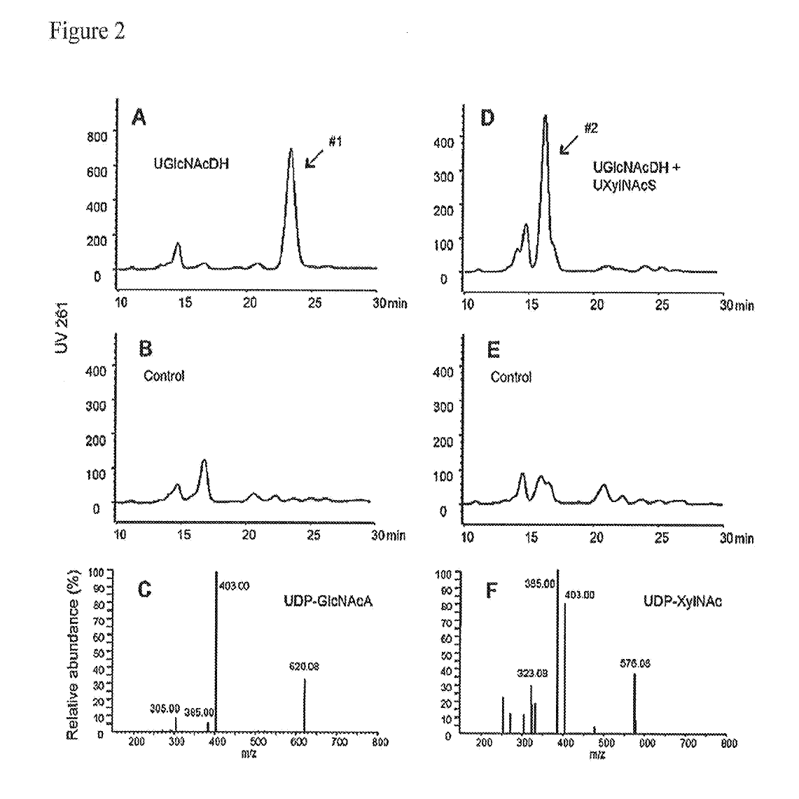

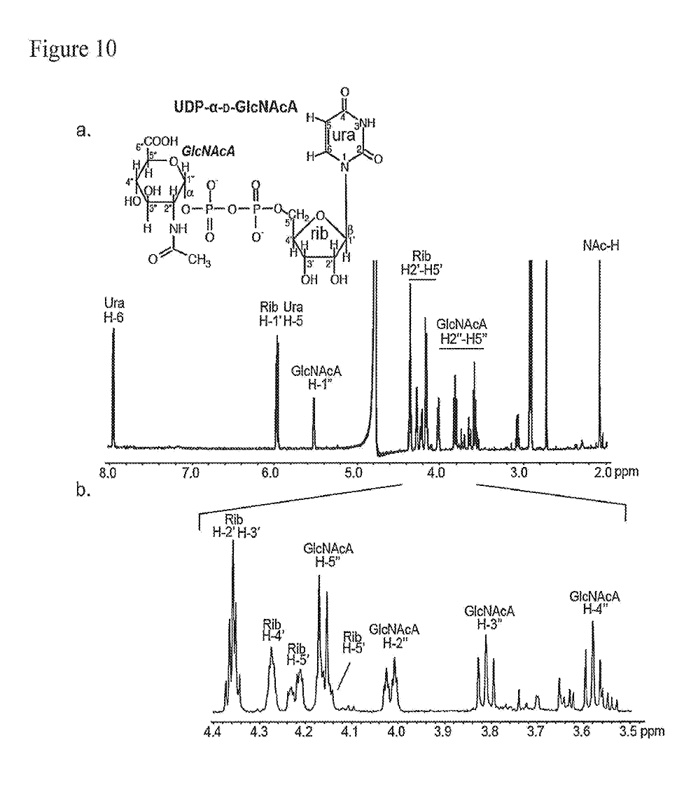

In one embodiment, cultures of E. coli harboring the UDP-GlcNAc dehydrogenase coding region were shown to generate a product that had a retention time of 23.2 min on anion-exchange chromatography (see FIG. 2 and compare panel A with panel B). The peak was collected and analyzed by LC-ESI-MS/MS (FIG. 2, panel C) and by ¹H-NMR (FIG. 10). The ESI-MS of the product contained a [M-H]⁻ ion at m/z 620.08, and CID (ms/ms) of this ion gave a predominant fragment ion at m/z 403.00, corresponding to de-protonated UDP. The chemical shifts of the products ¹H-NMR spectra (FIG. 10) are consistent with UDP-GlcNAcA.

E. coli produced approximately 12.5 μg/ml of UDP-GlcNAcA. This data suggests that the introduced coding region shunts the endogenously synthesized UDP-GlcNAc to UDP-GlcNAcA in amounts that do not have a discernible effect on the growth of the bacterium. It is possible higher yields of UDP-GlcNAc or UDP-GlcNAcA can be obtained by modifying certain culture conditions such as, for example, medium composition, temperature, and/or incubation times. In most experiments the cell pellet was suspended with NaF since a previous study[AB6] had suggested that this compound inhibits enzymes that degrade NDP-sugars. However, comparable yields of UDP-GlcNAcA were obtained if the bacterial cells were resuspended in water rather than NaF.

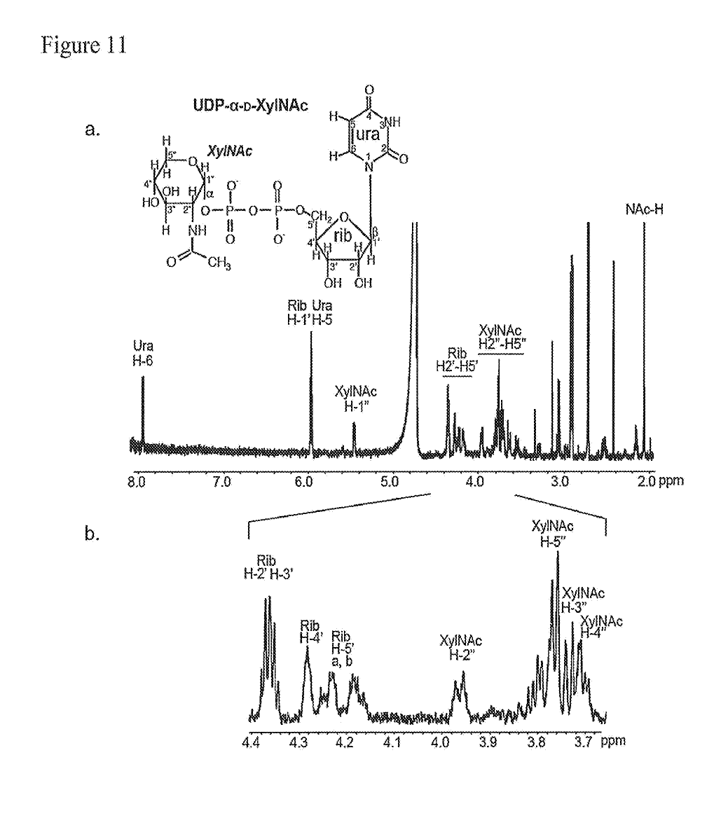

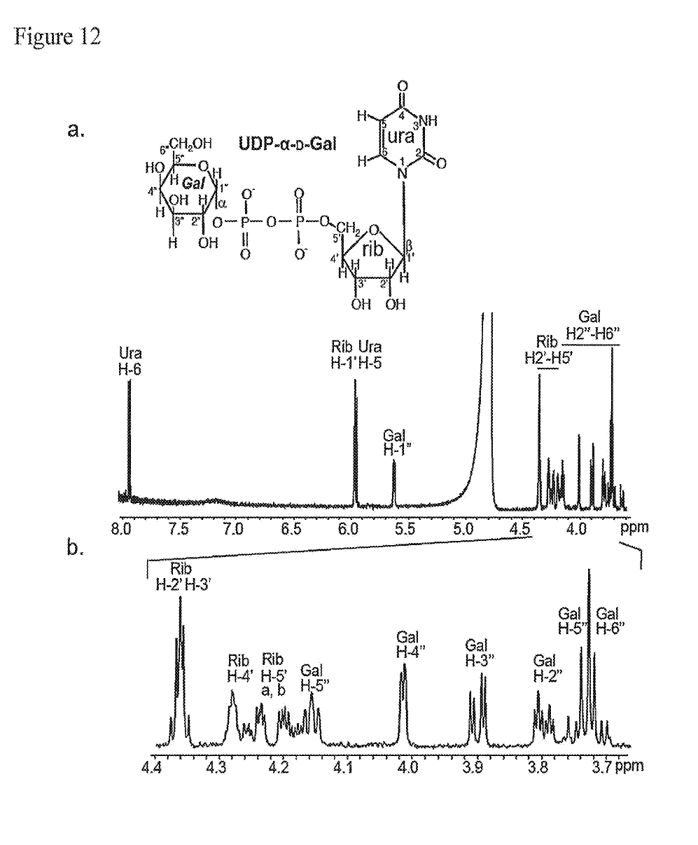

We next determined if E. coli could be engineered to produce UDP-XylNAc. For this purpose we introduced a single plasmid that independently drives the expression of the Bacillus cereus coding regions encoding UDP-GlcNAc dehydrogenase and UDP-XylNAc synthase. Aqueous extracts of cells harboring both coding regions (FIG. 2, panel D) but not the control cells (FIG. 2, panel E) gave a distinct peak (see arrow #2) when analyzed by HPLC. The ESI-MS of the collected peak (FIG. 2, panel F) contained a [M-H]⁻ ion at m/z of 576.08, corresponding to UDP-XylNAc (Gu et al., J Biol Chem (2010) 285:24825-24833). Negative ion MS/MS analysis of this ion fragment gave fragment ions at m/z 385.00 and 403.00, corresponding to [UDP-H-water]⁻ and [UDP-H]⁻, respectively. The ¹H-NMR spectrum of the product (FIG. 11) contained signals with chemical shifts characteristic of UDP-XylNAc (Gu et al., J Biol Chem (2010) 285:24825-24833). The yield of UDP-XylNAc was 5 μg/ml of culture. In this embodiment, the entirety of UDP-GlcNAcA appeared shunted to UDP-XylNAc.

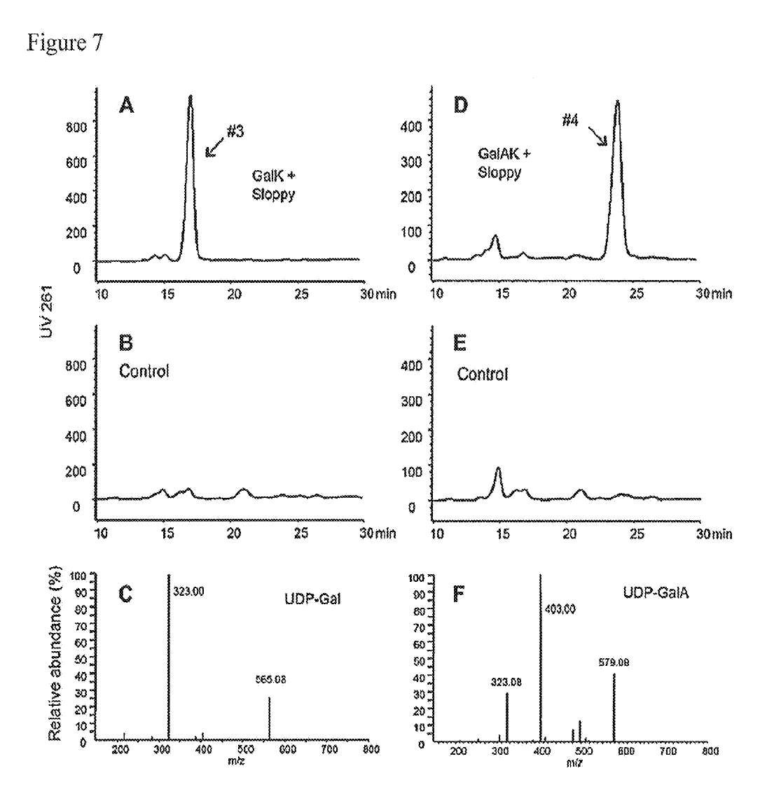

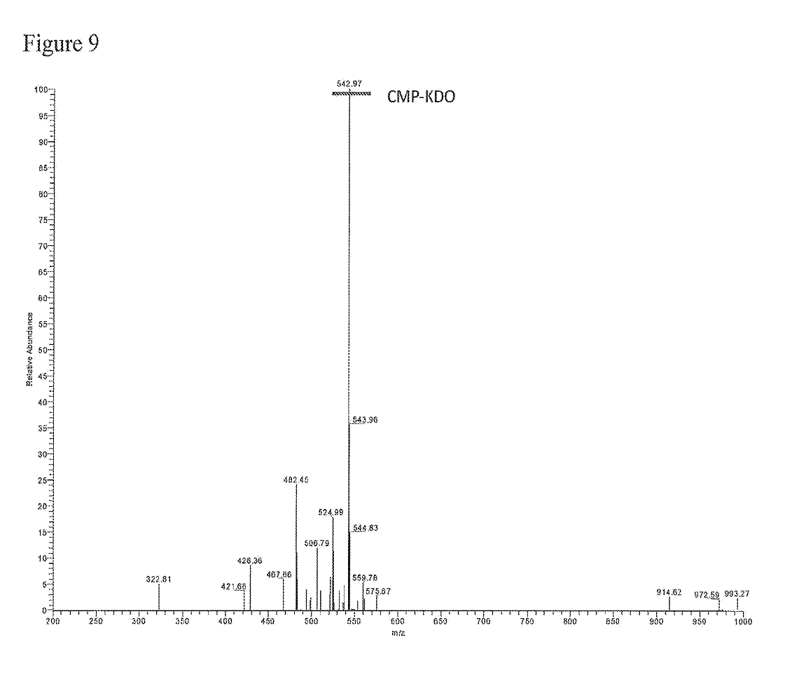

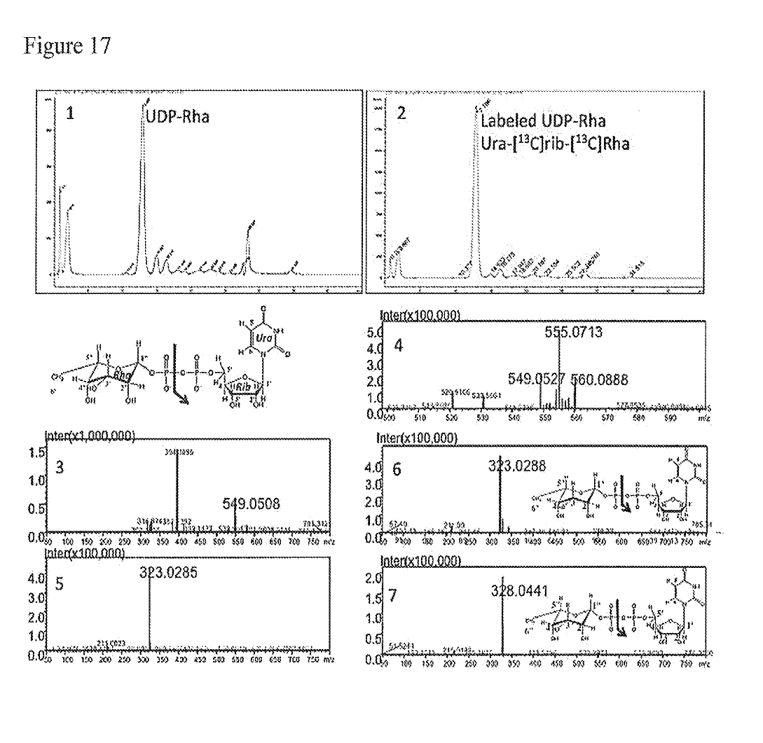

Thus, we have shown that an engineered cell can rapidly generate and accumulate UDP-GlcNAcA and UDP-XylNAc. Furthermore, while the exemplified embodiments involve transforming E. coli to express enzymes from B. cereus, the source of the enzyme and the encoding polynucleotide is not a limiting factor for the successful production of diverse NDP-sugars in our engineered biological system. We have successfully used the engineered biological system to produce NDP-sugars when the source of the coding regions used to transform a host cell was from a Gram-positive bacterium (e.g., Bacillus spp., see, e.g., FIG. 2), a Gram-negative bacterium (e.g., Escherichia spp., see FIG. 9), a fungus (e.g., Botryotinia spp. and Magnaporthe spp., see FIG. 17), and a plant (e.g., Arabidopsis spp., see FIG. 7). Alternatively, enzymes may be used from other sources such as, for example, Archaea (Mizanur et al., J. Am. Chem. Soc. (2004) 126:15993-15998), and from parasites (e.g., Trypanosoma; Yang and Bar-Peled, Biochem. J. (2010) 429:533-543).

### Isotope Labeling of UDP-GlcNAcA and UDP-XylNAc

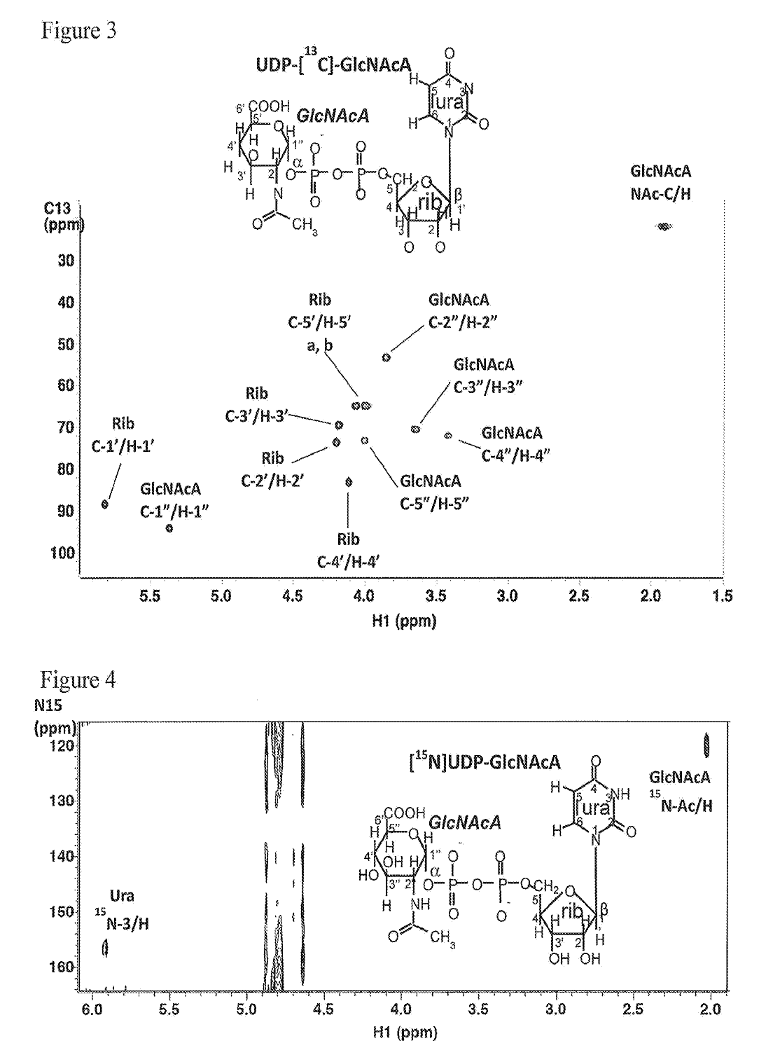

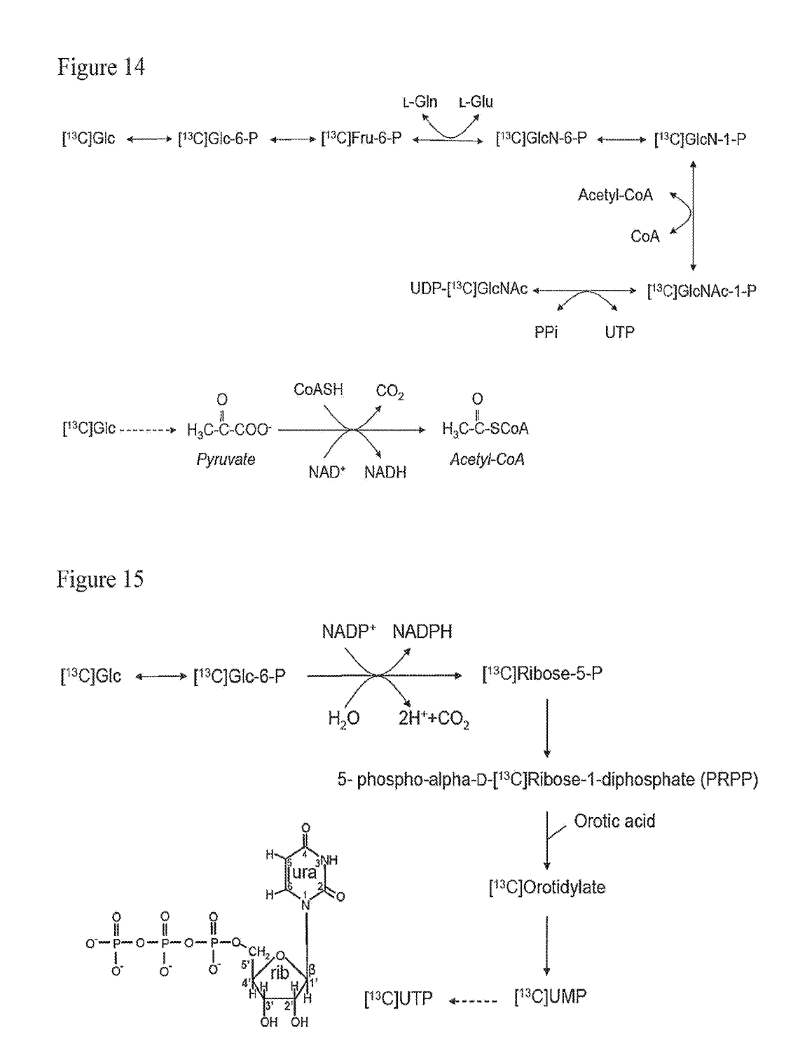

Next, we demonstrated that isotopically labeled UDP-sugars could be produced using our engineered biological system. We supplemented the growth media of E. coli harboring UDP-GlcNAc dehydrogenase with [U—¹³C]glucose. The NDP-sugars were extracted and the ¹³C signals were determined by NMR spectroscopy. A 2D ¹H—¹³C HSQC experiment (FIG. 3) showed that all six carbons of the GlcNAcA ring of UDP-GlcNAcA had a signal intensity consistent with ¹³C enrichment. Without wishing to be bound by any particular theory, the observed labeling may be due to the metabolic conversion of [¹³C]Glc to [¹³C]Glc6P and [¹³C]Frc6P, which give rise to [¹³C]glucosamine-1-P ([¹³C]GlcN-1-P), the precursor for UDP-[¹³C]GlcNAc (see metabolic scheme in FIG. 14).

The five carbons of the ribose moiety of UDP-GlcNAcA are also ¹³C-labeled. Again, without wishing to be bound by any particular theory, the observed labeling may be due to [¹³C]glucose coming from the pentose shunt generating D-[¹³C]ribose-5-P, the precursor for 5-phospho-α-D-[¹³C]ribose 1-diphosphate (PRPP). In E. coli, PRPP can be coupled with orotic acid to form orotidylate, which gives rise to UMP and UTP (FIG. 15). UTP along with GlcNAc-1-P form UDP-GlcNAc in E. coli.

The two carbons on the acetate group (NAc) of GlcNAcA moiety are also ¹³C-labeled. Again, without wishing to be bound by any particular theory, the observed labeling may be due to glycolysis I pathway (see illustrated metabolic pathway in FIG. 14), where glucose is converted to pyruvate. The pyruvate carbons can then be incorporated to the acetyl moiety of acetyl-CoA.

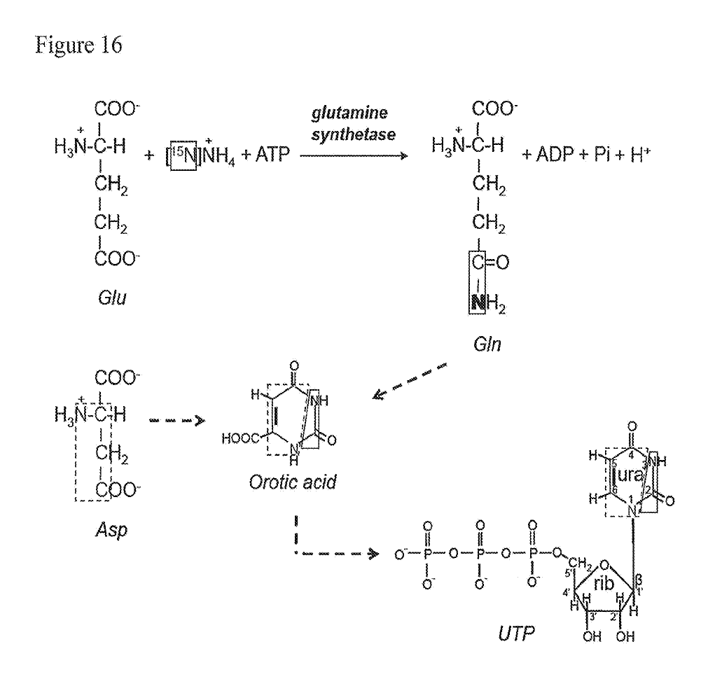

Interestingly, the uracil carbons of UDP-GlcNAcA are not ¹³C-labeled. This may be explained by metabolism leading to pyrimidine synthesis in E. coli. The C-2 and N-3 atoms in the pyrimidine ring come from carbamoyl phosphate, whereas the remaining atoms in the pyrimidine ring (N-1, C-6, C-5, and C-4) come from aspartate (see illustration in FIG. 16). A ID-¹H spectrum without ¹³C decoupling of labeled UDP-GlcNAcA showed the relative amount of ¹³C satellites to the central ¹²C peak to be over 90%, indicating that over 90% of UDP-GlcNAcA is labeled with ¹³C.

The uracil ring in the short labeling experiments described immediately above was not ¹³C-labeled. To determine if that ring can be labeled in the engineered biological system, we examined if ¹⁵N can be incorporated into UDP-GlcNAcA. For this purpose we fed E. coli with the precursor [¹⁵N]ammonium chloride. A 2D HMBC experiment (FIG. 4) demonstrated that the N-3 of the uracil ring and the nitrogen atom of the NAc-group of GlcNAcA moiety of UDP-GlcNAcA are selectively labeled with ¹⁵N, while N-1 is not ¹⁵N-labeled. ¹⁵N-Labeling of N-3 of the uracil ring may be explained by the incorporation of [¹⁵N]NH₃ to L-glutamine forming the carbamic acid and carbamoyl phosphate. ¹⁵N-Labeling of the nitrogen of N-acetyl-glucosaminuronic acid moiety may be explained by incorporation of the [¹⁵N]ammonia to L-glutamine (i.e., NH₃+phosphorylated glutamate) rather than its incorporation with ketoglutarate to glutamate (see metabolic scheme in FIG. 16).

Interestingly, the N-1 of the uracil ring of UDP-GlcNAcA was not labeled with ¹⁵N under the conditions described. To confirm this specific labeling of N-3, we fed the cell with [¹⁵N]L-glutamine. Both the N-3 of the uracil ring and the nitrogen atom of the NAc-group of GlcNAcA moiety of UDP-GlcNAcA were ¹⁵N-labeled. No ¹⁵N label was found of the nitrogen N-1 of uracil. In E. coli the N-1 is derived from the nitrogen group of aspartic acid (FIG. 16).

Additional proof that the N3, but not the N1, was labeled came from ¹⁵N HMBC acquired using a 21.1 Tesla magnet. At this field, the ribose H-1′ and the uracil H-5 are separated, and if Ni was labeled, one would expect to see a coupling to the ribose H-1′. However the ¹⁵N HMBC data confirmed that the observed ¹⁵N signal was coupled only to the uracil H-5 and not to the ribose H-1′. In addition, the coupling between uracil H-5 and H-6 to uracil Ni is to be very similar, but we obtained a strong signal coupled to H5 and a very weak signal to H6. This is more compatible with the three-bond N-3-H-5 and four-bond N-3-H-6 couplings. Taken together, we confirm that the ¹⁵N signal identified from uracil ring is on N-3.

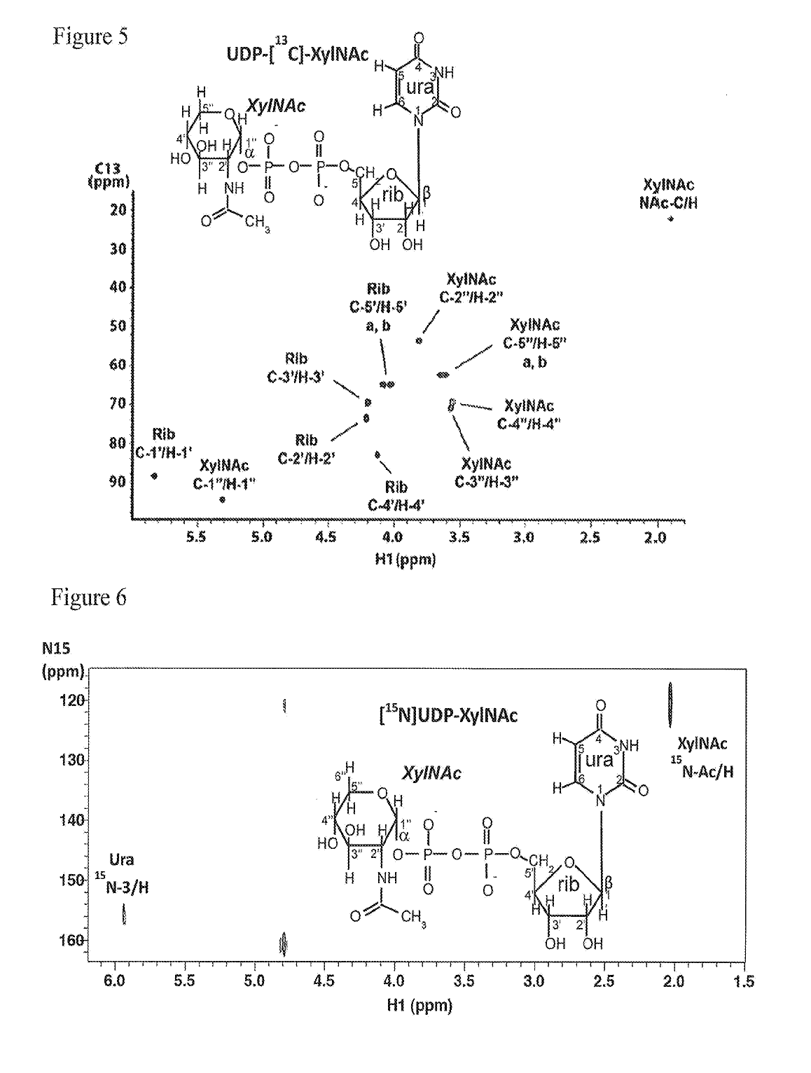

In a similar experiment we fed the E. coli strain that carries both UDP-GlcNAc dehydrogenase and UDP-XylNAc synthase coding regions with either [¹³C]glucose or the precursor [¹⁵N]NH₄Cl. 2D HSQC NMR experiments of the NDP-sugars isolated from cells grown in the presence of [U-¹³C]glucose revealed that ¹³C is in the carbons of the XylNAc and ribose but not, as found previously, in the uracil ring of UDP-XylNAc (FIG. 5). From the ID-¹H spectrum of labeled UDP-XylNAc, the relative amount of ¹³C satellites confirms over 95% ¹³C enrichment. Feeding E. coli with precursor [¹⁵N]ammonia again demonstrated that N-3 of the uracil ring and the nitrogen atom of the NAc group of XylNAc moiety of UDP-XylNAc are ¹⁵N, as shown in FIG. 6.

### Use of Engineered Biological System to Produce UDP-Gal and UDP-GalA

To determine if a recombinant cell could be engineered to accumulate other NDP-sugars, we introduced Arabidopsis coding regions encoding galactokinase (GalK) and UDP-sugar PPase (Sloppy) or galacturonic acid kinase (GalAK) and Sloppy[AB4] into E. coli. GalK in the presence of ATP converts α-D-galactose to α-D-Gal-1-P, which in the presence of Sloppy and UTP is converted to UDP-Gal (Yang et al., J. Biol. Chem. (2009) 284:21526-21535). In-vitro, the UDP-sugar PPase more effectively catalyzes the reverse reaction and unless PPi is depleted the reaction proceeds towards the formation of Gal-1-P rather than UDP-Gal. In vivo, however, pyrophosphate (PPi) derived from synthesis of DNA, RNA, and many other nucleotide metabolic pathways may be readily converted to 2Pi by phosphatases and PPiases. Hence, E. coli phosphatases may deplete PPi and drive the uridylylation reaction towards the formation of UDP-Gal.

The engineered bacteria cells harboring GalK and Sloppy coding regions were supplemented with galactose and accumulated a compound that eluted from HPLC column with a retention time of 16.8 min (see FIG. 7, panel A, marked by arrow #3). Control cells supplemented with Gal did not accumulate this compound (FIG. 7, panel B). Analysis of the compound by MS and MS/MS (FIG. 7, panel C) showed that the parent ion at m/z 565.08 is fragmented to an ion with m/z 323.00, suggesting that the product is a UDP-hexose and the ion fragment is UMP. ¹H-NMR analysis (FIG. 12) provides chemical shift values and coupling constants that are consistent with UDP-α-galactose (UDP-Gal). The E. coli line is deficient in GalK activity (galk). The yield of UDP-Gal in this in microbe system was 12.4 μg/ml.

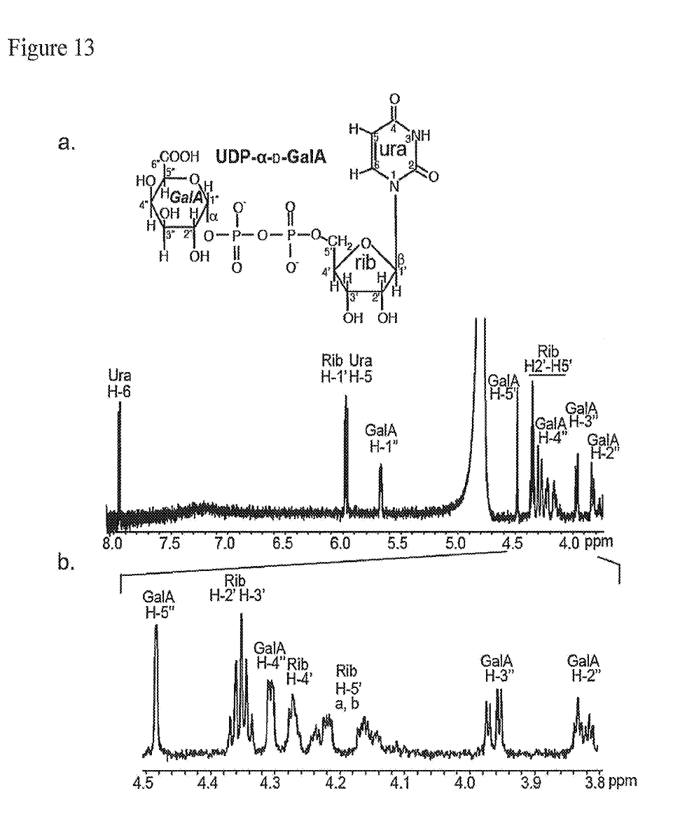

The next system we examined was E. coli engineered to contain GalAK and Sloppy coding regions grown in the presence of galacturonic acid. Cells in this system accumulated a product that eluted from the anion-exchange column with a retention time of 23.8 minutes (FIG. 7, panel D, arrow #4). No product was formed if no GalA was added. Analysis of the product by MS and MS/MS (FIG. 7 panel F) showed that the parent ion at m/z 579.08 is fragmented into two ions with m/z 403.00 and 323.08, suggesting that the product is a UDP-uronic acid and the ion fragments are [UDP-H]⁻ and [UMP-H]⁻, respectively. ¹H-NMR analysis (FIG. 13) gave chemical shifts and coupling constants that are consistent with UDP-α-galacturonic acid (UDP-GalA). The yield of UDP-GalA was 6.4 g/ml.

In alternative embodiments, Sloppy, a promiscuous UDP-sugar PPase (USP) from plants (Yang et al., J. Biol. Chem. (2009) 284:21526-21535; Litterer et al., Plant Physiol. Biochem. (2006) 44:171-180), can be replaced by other promiscuous NDP-sugar PPases from various species to convert diverse sugar-1-Ps to their corresponding UDP-sugars. For example, a UDP-sugar PPase from Trypanosoma cruzi converts Glc-1-P, Gal-1-P, Xyl-1-P and GlcA-1-P to their corresponding UDP-sugars (Yang and Bar-Peled, Biochem. J. (2010) 429:533-543). A bacterial RmlA that normally converts Glc-1-P and TTP to dTDP-Glc has been engineered to use multiple sugar-1-Ps as substrates (Moretti et al., J. Biol. Chem. (2011)286:13235-13243). Similarly, a promiscuous sugar nucleotidyltransferase from archaea has been used to form different UDP-sugars and dTTP-sugars (Mizanur et al., J. Am. Chem. Soc. (2004) 126:15993-15998).

### Towards Improving the Yields of UDP-Sugar

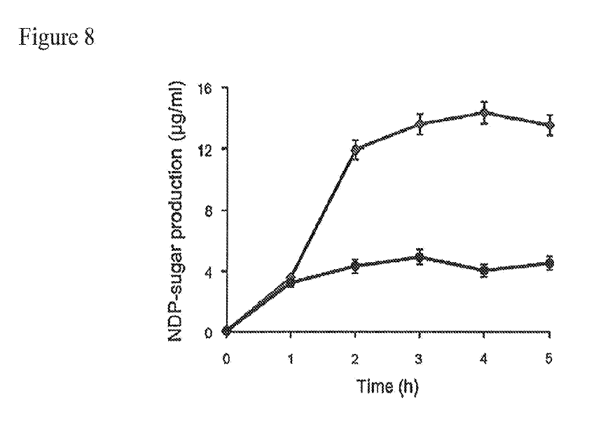

Next, we measured the time course of NDP-sugar production. The cells were grown in 20 ml of LB medium, and an aliquot (3 ml) was removed immediately after induction with IPTG (time 0) and then at hourly intervals for five hours. The NDP-sugars were extracted and quantified by HPLC and verify by LC-ESI-MS/MS. The results show that within two hours, the formation of NDP-sugar reached its maximum (FIG. 8). We then compared the amounts of NDP-sugars formed when E. coli was grown in flasks or in test tubes. The cells grew faster in the flask, possibly due to greater aeration, and produced between 20 and 30% more NDP-sugars than cells grown in test tubes (data not shown).

We have analyzed the requirement of sugars added for the NDP-sugar production. Engineered E. coli harboring GalAK and Sloppy produce UDP-GalA when provided exogenous GalA. Engineered E. coli harboring GalK and Sloppy produce a small amount of UDP-Gal in the absence of added galactose, which may be due to contamination by residual Gal in the rich media. However, a substantial (e.g., three-fold) increase in the production of UDP-Gal could be obtained by adding Gal to the growth media. E. coli harboring UDP-GlcNAcDH alone, or the combined activity of UDP-GlcNAcDH and UDP-XylNAc synthase, produces the corresponding UDP-GlcNAcA and UDP-XylNAc, respectively, without adding additional carbon sources to the rich media. Moreover, the yield is comparable to the yield of microbes fed with 0.2% Glc, Frc, or L-glutamine in the same media.

### A Comparison of Our Engineered Biological System and Other Methodologies for Preparing NDP-Sugars

Several procedures have been described for the biologically-based synthesis of nucleotide sugars including: i) in vitro enzymatic reactions (Moretti et al., J. Biol. Chem. (2011)286:13235-13243; Stein et al., Glycoconj. J. (1998) 15:139-145; Gantt et al., Nat. Prod. Rep. (2011) 28:1811-1853; Sugai et al., Bioorg. Med. Chem. (1995) 3:313-320); ii) in vitro synthesis of a NDP-sugar coupled with a glycosyltransferase engineered in bacteria, also known as a “one-pot reaction” (Mizanur et al., J. Am. Chem. Soc. (2005) 127:836-837; Hokke et al., Glycoconj. J. (1996) 13:687-692; Fu et al., Nat. Biotechnol. (2003)21:1467-1469; Hanson et al., Trends Biochem. Sci. (2004) 29:656-663); and iii) the engineered biological method described herein. Several groups have successfully generated milligram to gram amounts of UDP-Gal (Liu et al., Chem. Bio. Chem. (2002) 3:348-355; Butler and Elling, Glycoconj. J. (1999) 16:147-159; Heidlas et al., J. Org. Chem. (1992) 57:152-157), UDP-GalNAc (Heidlas et al., J. Org. Chem. (1992) 57:152-157), and radioactive UDP-GlcNAc (Lang and Kornfeld, Anal. Biochem. (1984) 140:264-269) using homogeneous enzymes. Azido-radioactive precursor (Drake et al., J. Biol. Chem. (1991) 266:23257-23260) of UDP-GlcA was also produced in vitro using a similar method. Each of these processes can be time-consuming and can require the addition of costly NAD⁺ and UDP-GlcNAc. Moreover, five additional recombinant enzymes (hexokinase, phosphoglucose isomerase, glutamine:Fru-6-P amidotransferase, GlcN phosphate mutase and glmU) can be required to generate ¹⁵N-labeled and ¹³C-labeled UDP-XylNAc and UDP-GlcNAcA using such in vitro methods. Expression plasmids harboring these five coding regions were not available to us, so we sought to develop an alternative in vivo system.

Using our engineered biological methods we showed that [¹³C]Glc and [¹⁵N]L-glutamine are readily incorporated into UDP-GlcNAcA and UDP-XylNAc (see FIGS. 3-6). In some embodiments, our engineered biological method can exploit the activity of endogenous enzymes to carry out the some enzymatic reactions. For example, some embodiments use E. coli as the recombinant host cell and can exploit the activity of endogenous enzymes, eliminating any need to supply an exogenous hexokinase, an exogenous phosphoglucose isomerase, an exogenous glutamine:Fru-6-P amidotransferase, an exogenous GlcN phosphate mutase or an exogenous glmU.

Purified recombinant enzymes often lose activity during storage and/or may become inactive. Using such partially active enzymes can result in decreased product yield. In contrast, our engineered biological system maintains enzymatic activity thereby alleviating such potential problems.

Butler and Elling (J. (1999) 16:147-159) have reviewed some of the disadvantages of using conventional in vitro enzymatic conversion. For example, the use of recombinant enzymes may not be successful in certain large-scale industrial processes. In contrast, our engineered biological methodology provides an efficient and convenient way to produce both normal and labeled NDP-sugars. In addition, it should be readily scalable to obtain NDP-sugars in amounts sufficient for small-scale research and/or large-scale industry use.

Currently, we are using the engineered biological system to generate many other nucleotide sugars including for example UDP-xylose, UDP-GlcA (see, Table 1). The system will enable to generate inexpensively and sufficient amount of other labeled derivatives such as deuterium-labeled NDP-sugars, radioactive NDP-sugars, or modified NDP-sugars (e.g., azido-sugar nucleotide and deoxy-sugar nucleotide) that are critical for glycobiology research. The system also provides means to evaluate biosynthetic pathways and determine how precursors enter different metabolic pathways.

TABLE 1

| Coding region(s) added to | Gene seq ID (amino | Enzyme Commission | | supplement added | activated sugar nucleotide |
|---|---|---|---|---|---|
| cell | acid) | Number | notes | to medium | produced (full name) |
| UDP-GlcNAc | GU784842.1 | 1.1.1.136 | | None required, but | UDP-GlcNAcA (UDP-N- |
| dehydrogenase | | | | improved yield can | acetylglucuronic acid) |
| (UGlcNAcDH) | | | | be obtained with | |
| | | | | selected carbon | |
| GalA kinase | GalAK: At3g10700 | 2.7.1.44 (GalAK); | | GalA | UDP-GalA (UDP- |
| and UDP-sugar | Sloppy: At5g52560 | 2.7.7.64 (sloppy) | | | galacturonic acid) |
| pyrophosphorylase | | | | | |
| (GalAK + sloppy) | | | | | |
| GalA kinase mutant | GalAK^{Y250F}: | 2.7.1.44 (GalAK); | This mutation | GlcA | UDP-GlcA (UDP-glucuronic |
| (Y250F) and UDP-sugar | At3g10700^{Y2S0F} | 2.7.7.64 (sloppy) | enables the | | acid) |
| pyrophosphorylase | Sloppy: At5g52560 | | enzyme to utilize | | |
| (GalAK^{Y250F} + sloppy) | | | GlcA | | |
| Gal kinase and UDP-sugar | GalK: At3g06580 | 2.7.1.6 (GalK); | | Gal | UDP-Gal (UDP-galactose) |
| pyrophosphorylase | Sloppy: At5g52560 | 2.7.7.64 (sloppy) | | | |
| (GalK + sloppy) | | | | | |
| Gal kinase and UDP-sugar | GalK: At3g06580 | 2.7.1.6 (GalK); | Sugar derivative | 2-deoxy-Gal | UDP-2-deoxy-Gal (UDP-2- |
| pyrophosphorylase | Sloppy: At5g52560 | 2.7.7.64 (sloppy) | | | deoxy-galactose) |
| (GalK + sloppy) | | | | | |
| Gal kinase mutant (S206G) | GalK: | 2.7.1.6 (GalK); | The mutation of | GalNAc | UDP-GalNAc (UDP-N- |
| and N-acetylgalactosamine- | At3g06580^{S206G} | 2.7.7.23 | GalK enables the | | acetyl-galactosamine) |
| 1-phosphate | GalNAc1pUT1: | (GalNAc1pUT) | enzyme to utilize | | |
| uridylyltransferase, | At1g31070 | | GalNAc | | |
| (GalK^{S206G} + GalNAc1pUT) | GalNAc1pUT2: | | | | |
| | At2g35020 | | | | |
| UDP-Glc PPase or | UGlcPPase1: | 2.7.7.9 (UGPPase); | | None required, but | UDP-Glc (UDP-glucose) |
| UDP-sugar PPase (Sloppy) | At5g17310 | 2.7.7.64 (sloppy) | | improved yield can | |
| | UGlcPPase2: | | | be obtained with | |
| | At3g03250 | | | selected carbon | |
| | Sloppy: At5g52560 | | | | |
| UDP-GlcA decarboxylase | UXS1: At3g53520 | 4.1.1.35 | | None required, but | UDP-Xyl (UDP-xylose) |
| (UXS) | UXS2: At3g62830 | | | improved yield can | |
| | UXS3: At2g47650 | | | be obtained with | |
| | UXS4: At5g59290 | | | selected carbon | |
| | UXS5: At3g46440 | | | | |
| | UXS6: At2g28760 | | | | |
| UDP-GlcNAc | GU784842.1 | 1.1.1.136 | | None required, but | UDP-XylNAc (UDP-2- |
| dehydrogenase and UDP- | (UGlcNAcDH) | (UGlcNAcDH) | | improved yield can | acetamido-2-deoxy-xylose) |
| XylNAc synthase | GU784843.1 | | | be obtained with | |
| (UGlcNAcDH + UXNAcS) | (UXNAcS) | | | selected carbon | |
| UDP-glucose-4,6- | AEH41993.1, | 4.2.1.76 | | None required, but | UDP-4keto-6deoxyglucose |
| dehydratase (UG4,6-DH) | AEH41995.1 | | | improved yield can | |
| | | | | be obtained with | |
| | | | | selected carbon | |
| UDP-glucose-4,6- | AEH41993.1 | 4.2.1.76 (UG4,6-DH) | | None required, but | UDP-Rha (UDP-rhamnose) |
| dehydratase and UDP-4- | (UG4,6-DH) | | | improved yield can | |
| keto-6-deoxyglucose-3,5- | AEH41994.1 | | | be obtained with | |
| epimerase/-4-reductase | (U4k6dG-ER) | | | selected carbon | |
| (UG4,6-DH + U4k6dG-ER) | AEH41996.1 | | | | |
| UDP-4-keto-pentose/UDP- | AY057445 | 1.1.1.305 | | None required, but | UDP-4-keto-xylose |
| xylose Synthase | | | | improved yield can | |
| (U4kpxs) | | | | be obtained with | |
| | | | | selected carbon | |
| UDP-glucose-4,6- | AEH41993.1 | 4.2.1.76 | | None required, but | UDP-Rha (UDP-rhamnose) |
| dehydratase (UG4,6-DH) | | | | improved yield can | |
| | | | | be obtained with | |
| | | | | selected carbon | |
| UDP-Apiose synthase | ABC75032.1 | | | None required, but | UDP-Api (UDP-apiose) |
| (UApiS) | | | | improved yield can | |
| | | | | be obtained with | |
| | | | | selected carbon | |
| CMP-KDO synthase | AAA83877.1 | 2.7.7.38 | | None required, but | CMP-KDO (CMP-3-deoxy- |
| (CKdoS) | | | | improved yield can | manno-octulosonate) |
| | | | | be obtained with | |
| | | | | selected carbon | |
| CMP-KDO synthase | AAA83877.1 | 2.7.7.38 | Azido- sugar | ^{$}KDO-N₃ | CMP-KDO-N3 (Azido-CMP- |
| (CKdoS) | | | derivative | | 3-deoxy-manno-octulosonate) |
| CMP-DHA synthase | N/A | N/A | | None required, but | DHA (3-deoxy-D-lyxo- |
| (CDhaS) | | | | improved yield can | heptulosaric acid) |
| | | | | be obtained with | |
| | | | | selected carbon | |
| Galacturonic acid kinase | GalAK: At3g10700 | 2.7.1.44 | | GalA | GalA-1-P (galacturonic acid- |
| (GalAK) | | | | | 1-phosphate) |
| Galacturonic acid kinase | GalAK: | 2.7.1.44 | This mutation | GlcA | GlcA-1-P (glucuronic acid-1- |
| mutant (Y250F) | At3g10700^{Y250F} | | will enable the | | phosphate) |
| (GalAK^{Y250F}) | | | enzyme to utilize | | |
| | | | GlcA | | |
| Galactose kinase (GalK) | GalK: At3g06580 | 2.7.1.6 | | Gal | Gal-1-P (galactose-1-P) |
| Galactose kinase | GalK: At3g06580 | 2.7.1.6 | Sugar derivative | 2-deoxy-Gal | 2d-Gal-1-P (2-deoxy- |
| (GalK) | | | | | galactose-1-phosphate) |
| Galactose kinase mutant | GalK: | 2.7.1.6 | This mutation | GalNAc | GalNAc-1-P (N-acetyl- |
| (S206G) | At3g06580^{S206G} | | will enable the | | galactosamine-1-P) |
| (GalK^{S206G}) | | | enzyme to utilize | | |
| | | | GalNAc | | |
| N-acetylglucosamine-1- | GlcNAc1pUT1: | 2.7.7.23 | | None required, but | UDP-GlcNAc (UDP-N- |
| phosphate | At1g31070 | | | improved yield can | acetylglucosamine |
| uridylyltransferase, | GlcNAclpUT2: | | | be obtained with | |
| (GlcNAc1pUT) | At2g35020 | | | selected carbon | |
| UDP-glucuronic acid 4- | UGlcAE1: | 5.1.3.6 | | None required, but | UDP-GalA (UDP- |
| epimerase | At2g45310 | | | improved yield can | galacturonic acid) |
| (UGlcAE) | UGlcAE2: | | | be obtained with | |
| | At3g23820 | | | selected carbon | |
| | UGlcAE3: | | | | |
| | At4g30440 | | | | |
| mannose-1-phosphate | GMPPase1: | 2.7.7.13 | | None required, but | GDP-Man (GDP-mannose) |
| guanosylyltransferase, | At2g39770 | | | improved yield can | |
| (GDP-Man PPase) | GMPPase2: | | | be obtained with | |
| | At3g55590 | | | selected carbon | |
| UDP-glucose 6- | AF405548.1 | 1.1.1.22 | | None required, but | UDP-GlcA (UDP-glucuronic |
| dehydrogenase | | | | improved yield can | acid) |
| (UGDH) | | | | be obtained with | |
| | | | | selected carbon | |
| α-glucan phosphorylase + | AAE78225 (αGPho) | 2.4.1.— (αGPho); | | Starch or glycogen, | UDP-Glc (UDP-glucose) |
| UDP-Glc PPase | UGlcPPase1: | 2.1.1.9 (UGPPase) | | maltose | |
| (aGPho + UGPpase) | At5g17310 | | | | |
| | UGlcPPase2: | | | | |
| | At3g03250 | | | | |
| UDP-xylose 4-epimerase + | ADK79128 (UXE) | 5.1.3.5 (UXE); | | None required, but | UDP-Ara |
| UDP-glucose 6- | AF405548.1 (UGDH) | 1.1.1.22 (UGDH); | | improved yield can | (UDP-arabinopyranose) |
| dehydrogenase + | At3g53520 (UXS) | 4.1.1.35 (UXS) | | be obtained with | |
| UDP-GlcA | | | | selected carbon | |
| decarboxylase | | | | | |
| (UXE + UGDH + UXS) | | | | | |
| UDP-arabino mutase + | XP_002325896 | 5.4.99.30 (UAraM); | | None required, but | UDP-Araf |
| UDP-xylose 4-epimerase + | (UAraM) | 5.1.3.5 (UXE); | | improved yield can | (UDP-arabinofuranose) |
| UDP-glucose 6- | ADK79128 (UXE) | 1.1.1.22 (UGDH) | | be obtained with | |
| dehydrogenase + | AF405548.1 (UGDH) | 4.1.1.35 (UXS) | | selected carbon | |
| UDP-GlcA | At3g53520 (UXS) | | | | |
| decarboxylase | | | | | |
| (UAraM + UXE + UGDH + | | | | | |
| UXS) | | | | | |
| Sucrose synthase and UDP- | CAA43303.1 (Susy) | 2.4.1.13 (Susy); | | sucrose | UDP-Rha (UDP-Rhamnose) |
| Rhamnose synthase | AT1G63000 (URS) | 2.4.1.— (URS) | | | |
| (Susy + URS) | | | | | |
| Sucrose synthase and UDP- | CAA43303.1 (Susy) | 2.4.1.13 (Susy); | | sucrose | UDP-GlcA |
| Glc-6 dehydrogenase | AF405548.1 (UGDH) | 1.1.1.22 (UGDH) | | | (UDP-glucuronic acid) |
| (Susy + UGDH) | | | | | |
| Sucrose synthase and UDP- | CAA43303.1 (Susy) | 2.4.1.13 (Susy); | | sucrose | UDP-Gal (UDP-galactose) |
| Glc-4-epimerase | AAE34241.1 | 5.1.3.2 (UGlcE) | | | |
| (Susy + UGlcE) | (UGlcE) | | | | |
| Sucrose synthase + UDP- | CAA43303.1 (Susy) | 2.4.1.13 (Susy); | | sucrose | UDP-Xyl (UDP-xylose) |
| Glc-6 dehydrogenase + | AF405548.1 (UGDH) | 1.1.1.22 (UGDH); | | | |
| UDP-GlcA decarboxylase | At3g53520 (UXS) | 4.1.1.35 (UXS) | | | |
| (Susy + UGDH + UXS) | | | | | |
| α-glucan phosphorylase + | AAE78225 (αGPho) | 2.4.1.— (αGPho); | | Starch or glycogen, | UDP-Rha (UDP-rhamnose) |
| UDP-Glc PPase + UDP- | UGlcPPase1: | 2.1.1.9 (UGPPase); | | maltose | |
| rhamnose synthase | At5g17310 | 2.4.1.— (URS) | | | |
| (αGPho + UGPpase + URS) | UGlcPPase2: | | | | |
| | At3g03250 | | | | |
| | AT1G63000 (URS) | | | | |
| GDP-Man 3,5-epimerase | A3C4S4.1 | 5.1.3.18 | | None required, but | GDP-L-Gal |
| (GME) | | | | improved yield can | (GDP-L-galactose) |
| | | | | be obtained with | |
| | | | | selected carbon | |
| GDP-Man PPase + | GMPPase1: | 2.7.7.13 (GMP); | | None required, but | GDP-Fuc (GDP-fucose) |
| GDP-Man4,6DH + | At2g39770 | 4.2.1.47 (GDP- | | improved yield can | |
| GDP-4keto-6dMan, 3,5- | GMPPase2: | Man4,6DH); | | be obtained with | |
| epimerase/4-reductase | At3g55590 | 5.1.3.18 (G3,5ER) | | selected carbon | |
| | A3C4S4.1 (GME) | | | | |
| Bacterial | N/A | | Hungati 832 | None required, but | UDP-sugar-Me |
| NDP-sugar-C- | | | | improved yield can | (methylated nucleotide sugar) |
| Methyltransferase | | | | be obtained with | |
| | | | | selected carbon | |

### Genetically Engineered Cells

Thus, in one aspect, this disclosure provides genetically engineered cells. Generally, the genetically engineered cells exhibit an increase in synthesis of an activated sugar-nucleotide compared to a wild type control. The increase may be manifested as either a measurable increase in the amount of one or more NDP-sugars that are native to the host and/or production of one or more nucleotide-sugars (and/or derivatives thereof) that are not native to the host (e.g., UDP-XylNac, UDP-GalA, UDP-GlcA).

An increase in the synthesis of an activated sugar-nucleotide can be quantitatively measured and described in terms of a percentage of the functional activity of a comparable control such as, for example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 110%, at least 125%, at least 150%, at least 200%, or at least 250% greater than the activity of a suitable control. In circumstances in which the suitable control does not produce any measurable amount of a particular activated sugar-nucleotide, any measurable synthesis of the activated sugar-nucleotide by the genetically engineered cell reflects an increase in the synthesis of the activated sugar-nucleotide.

As used herein, a “genetically engineered cell” refers to a cell that includes a polynucleotide that it does not naturally possess. For example, a cell can be a genetically modified cell because an exogenous polynucleotide has been introduced into the cell. A “genetically modified cell” also can refer to a cell that has been genetically manipulated such that at least one endogenous nucleotide has been altered. Thus, one example of a genetically modified cell is a cell having an altered regulatory sequence such as, for example, a promoter that results in an increase or a decrease in expression of an endogenous coding region operably linked to the promoter.

As used herein, the term “polynucleotide” refers to a polymeric form of nucleotides of any length, either ribonucleotides or deoxynucleotides, and includes both double-stranded and single-stranded DNA and RNA. A polynucleotide may include nucleotide sequences having different functions such as, for example, coding sequences and/or and non-coding sequences such as, for example, regulatory sequences. A polynucleotide can be obtained directly from a natural source or can be prepared with the aid of recombinant, enzymatic, or chemical techniques. A polynucleotide can be linear or circular in topology. A polynucleotide can be, for example, a vector such as an expression vector or a cloning vector, or a fragment thereof.

“Coding sequence” or “coding region” refers to a nucleotide sequence that encodes a polypeptide and, when placed under the control of appropriate regulatory sequences, expresses the encoded polypeptide. The boundaries of a coding region are generally determined by a translation start codon at its 5′ end and a translation stop codon at its 3′ end. As used herein, the term “polypeptide” refers broadly to a polymer of two or more amino acids joined together by peptide bonds. The term “polypeptide” also includes molecules that contain more than one polypeptide joined by disulfide bonds, ionic bonds, or hydrophobic interactions, or complexes of polypeptides that are joined together, covalently or noncovalently, as multimers (e.g., dimers, tetramers). Thus, the terms peptide, oligopeptide, and protein are all included within the definition of polypeptide and these terms are used interchangeably. The term “polypeptide” does not connote a specific length of a polymer of amino acids, nor does it imply or distinguish whether the polypeptide is produced using recombinant techniques, chemical or enzymatic synthesis, or is naturally occurring.

“Regulatory sequence” refers to a nucleotide sequence that regulates expression of a coding region to which it is operably linked. Nonlimiting examples of regulatory sequences include, for example, promoters, transcription initiation sites, translation start sites, translation stop sites, and terminators. “Operably linked” refers to a juxtaposition wherein the components are in a relationship permitting them to function in their intended manner. A regulatory sequence is “operably linked” to a coding region when it is joined in such a way that expression of the coding region is achieved under conditions compatible with the regulatory sequence.

“Exogenous polynucleotide” refers to a foreign polynucleotide (i.e., a polynucleotide that is not normally present in a cell) or a polynucleotide that is normally present in a cell but includes a coding region that is operably linked to a regulatory region to which it is not normally operably linked. Such a regulatory region may be (a) a regulatory region not normally present in the cell, (b) a regulatory region normally present in the cell but not normally operably linked to the coding sequence encoding a polypeptide involved in production of an activated sugar-nucleotide, and/or (c) a regulatory region modified to increase expression of a coding region operably linked to the regulatory region.

A genetically engineered cell described herein includes at least one exogenous polynucleotide encoding a polypeptide involved in the production of an activated sugar-nucleotide. That is, a polynucleotide that encodes a polypeptide involved in the production of an activated sugar-nucleotide that is either not natively present in the cell or is under the control of a regulatory region to which it is not normally operably linked.

In one embodiment, the activated sugar-nucleotide is one that is not made by a control cell. As used herein, a “control” cell is one that is genetically similar to a genetically engineered cell, but does not include the exogenous polynucleotide present in the genetically engineered cell and/or does not include the genetically manipulated endogenous nucleotides present in the genetically engineered cell. In another embodiment, the genetically engineered cell can be engineered to overexpress one or more native polypeptides in the production of an activated sugar-nucleotide so that the genetically engineered cell exhibits an increase in synthesis of an activated sugar-nucleotide compared to a wild type control.

The genetically engineered cell can be, or be derived from, any suitable host cell including, for example, a prokaryotic microbe or a eukaryotic cell. As used herein, the term “or derived from” in connection with a host cell simply allows for the host cell to possess one or more genetic modifications before being modified to include a exogenous polynucleotide that encodes a polypeptide that is involved in the production of an activated sugar-nucleotide. Thus, the term “genetically engineered cell” encompasses a host cell that may contain nucleic acid material from more than one species before having the exogenous polynucleotide that encodes a polypeptide that is involved in the production of an activated sugar-nucleotide introduced into the cell.

In some embodiments, the genetically engineered cell may be, or be derived from, a eukaryotic microbe or eukaryotic multicellular organism such as, for example, a fungal cell, a human cell, a parasite cell, a plant cell, etc. In some of these embodiments, the fungal cell may be, or be derived from, a member of the Saccharomycetaceae family such as, for example, Saccharomyces cerevisiae, a member of the genus Candida such as, for example, Candida albicans, a member of the genus Kluyvermyces, or a member of the genus Pichia such as, for example, Pichia pastoris. In other embodiments, the fungal cell may be a member of the family Dipodascaceae such as, for example, Yarrowia lipolytica, a member of the genus Botryotinia (e.g., Botryotinia fuckeliana) including anamorphs (e.g., Botrytis cinerea), a member of the genus Magnaporthe (e.g., Magnaporthe oryzae), or a member of the genus Neurospora (e.g., Neurospora crassa). In some embodiments, the genetically engineered cell may be, or be derived from, a cell from a member of the genus Trypanosoma (e.g., Trypanosoma cruzi, T. brucei, etc.).

In other embodiments, the genetically engineered cell may be, or be derived from, a prokaryotic microbe such as, for example, a bacterium. In some of these embodiments, the bacterium may be a member of the phylum Protobacteria. Exemplary members of the phylum Protobacteria include, for example, members of the Enterobacteriaceae family (e.g., Escherichia coli) and, for example, members of the Pseudomonaceae family (e.g., Pseudomonas putida). In other cases, the bacterium may be a member of the phylum Firmicutes. Exemplary members of the phylum Firmicutes include, for example, members of the Bacillaceae family (e.g., Bacillus subtilis, B. cereus, B. thuringiensis, etc.) and, for example, members of the Streptococcaceae family (e.g., Lactococcus lactis). In certain particular embodiments, the genetically engineered cell can include E. coli, B. subtilis, B. thuringiensis, or other GRAS-like species.

In some embodiments, the genetically engineered cell may be engineered to include an exogenous polynucleotide from a eukaryotic cell. The eukaryotic cell may be, or be derived from, a eukaryotic microbe such as, for example, a fungal cell. Alternatively, the eukaryotic cell may be, or be derived from, a cell from a multicellular eukaryotic organism such as, for example, a plant or an animal. In some of these embodiments, the fungal cell may be, or be derived from, a member of the Saccharomycetaceae family such as, for example, Saccharomyces cerevisiae, a member of the genus Candida such as, for example, Candida albicans, a member of the genus Kluyvermyces, or a member of the genus Pichia such as, for example, Pichia pastoris. In other embodiments, the fungal cell may be a member of the family Dipodascaceae such as, for example, Yarrowia lipolytica, a member of the genus Botryotinia (e.g., Botryotinia fuckeliana) including anamorphs (e.g., Botrytis cinerea), a member of the genus Magnaporthe (e.g., Magnaporthe oryzae), or a member of the genus Neurospora (e.g., Neurospora crassa). In other embodiments, the eukaryotic cells may be, or be derived from, a plant such as, for example, a member of the genus Arabidposis, a woody plant such as, for example, a Populus spp., or a grass such as, for example, rice or switchgrass. In other embodiments, the eukaryotic cell may be, or be derived from, a cell from an animal such as, for example, a member of the genus Trypanosoma (e.g., Trypanosoma cruzi, T. brucei, etc.) or a member of the genus Homo (e.g., Homo sapiens).

In other embodiments, the genetically engineered cell may be engineered to include an exogenous polynucleotide from a prokaryotic microbe such as, for example, a bacterium. In some of these embodiments, the bacterium may be a member of the phylum Protobacteria. Exemplary members of the phylum Protobacteria include, for example, members of the Enterobacteriaceae family (e.g., Escherichia coli), members of the Pseudomonaceae family (e.g., Pseudomonas putida), and, for example, members of the Rhizobiales Family (e.g., Sinorhizobium meliloti, Rhizobium leguminosarum, Agrobacterium spp.). In other cases, the bacterium may be a member of the phylum Firmicutes. Exemplary members of the phylum Firmicutes include, for example, members of the Bacillaceae family (e.g., Bacillus subtilis, B. cereus, B. thuringiensis, etc.) and, for example, members of the Streptococcaceae family (e.g., Lactococcus lactis).

In some embodiments, the activated sugar-nucleotide produced by the genetically engineered cell may be a uridine diphosphate (UDP) sugar-nucleotide. Examples of uridine diphosphate sugar-nucleotides include, but are not limited to, UDP-galactose (UDP-Gal), UDP-galacturonic acid (UDP-GalA), UDP-N-acetylglucuronic acid (UDP-GlcNAcA), and UDP-2-acetamido-2-deoxy-xylose (UDP-XylNAc). Other examples of UDP sugar-nucleotides are disclosed in Table 1. In one embodiment, the nucleotide sugar produced by the cell uses a sugar that is imported into the genetically engineered cell and then joined to a UDP. Such UDP sugar-nucleotides include, but are not limited to, UDP-Gal (where the galactose is imported into the cell), UDP-GalA (where the galacturonic acid is imported into the cell), and UDP-glucuronic acid (where the glucuronic acid is imported into the cell).

In some embodiments, the activated sugar-nucleotide produced by the genetically engineered cell may be a cysteine monophosphate (CMP) sugar-nucleotide. Examples of cysteine monophosphate sugar-nucleotides include, but are not limited to, CMP-3-deoxy-manno-octulosonate (CMP-KDO) and azido-CMP-3-deoxy-manno-octulosonate (CMP-KDO-N₃). In one embodiment, the nucleotide sugar produced by the cell uses a sugar that is imported into the genetically engineered cell and then joined to a CMP. An example of such a CMP sugar-nucleotide includes, but is not limited to, CMP-KDO-N₃) (where the azido-sugar derivative KDO-N₃ is imported into the cell.

In some embodiments, the activated sugar-nucleotide produced by the genetically engineered cell may be a guanosine diphosphate (GDP) sugar-nucleotide. Examples of guanosine diphosphate sugar-nucleotides include, but are not limited to, GDP-mannose (GDP-Man) and GDP-L-galactose (GDP-L-Gal).

In some embodiments, the activated sugar-nucleotide can include an isotopic label, which may or may not be radioactive. Exemplary labeling isotopes that may be incorporated into an activated sugar-nucleotide include, for example, ²H, ¹³C, ¹⁴C, ¹⁵N, ¹⁷O, ¹⁸, ³²P, or ³³P.

Exemplary polynucleotides that encode a polypeptide that results in the production of an activated sugar-nucleotide by a genetically engineered cell into which one or more of the polynucleotides is introduced are identified in Table 1. A polynucleotide encoding a polypeptide can be inserted in a vector. A vector is a replicating polynucleotide, such as a plasmid, phage, or cosmid, to which another polynucleotide may be attached so as to bring about the replication of the attached polynucleotide. Construction of vectors containing a polynucleotide of the invention employs standard ligation techniques known in the art. See, e.g., Sambrook et al, Molecular Cloning: A Laboratory Manual., Cold Spring Harbor Laboratory Press (1989). A vector can provide for further cloning (amplification of the polynucleotide), i.e., a cloning vector, or for expression of the polypeptide encoded by the coding region, i.e., an expression vector. The term vector includes, but is not limited to, plasmid vectors, viral vectors, cosmid vectors, or artificial chromosome vectors. In one embodiment, the vector is a plasmid.

Selection of a vector can depend upon a variety of desired characteristics in the resulting construct, such as a selection marker, vector replication rate, and the like. Suitable host cells for cloning or expressing the vectors herein are prokaryote or eukaryotic cells. Preferably the host cell secretes minimal amounts of proteolytic enzymes. Suitable prokaryotes include eubacteria, such as gram-negative organisms, for example, E. coli or S. typhimurium.

An expression vector optionally includes regulatory sequences operably linked to the coding region. The invention is not limited by the use of any particular promoter, and a wide variety of promoters are known. Promoters act as regulatory signals that bind RNA polymerase in a cell to initiate transcription of a downstream (3′ direction) coding region. The promoter used can be a constitutive or an inducible promoter. It can be, but need not be, heterologous with respect to the host cell. Examples of promoters include, but are not limited to, trp, tac, and T7.

### Methods of Use

In another aspect, this disclosure also provides methods for using the genetically engineered cells described herein. In some embodiments, the method can include producing a sugar-nucleotide such as, for example, a UDP sugar-nucleotide, a CMP sugar-nucleotide, a GDP sugar-nucleotide, an ADP sugar-nucleotide, and/or one of the other activated sugar-nucleotides described in Table 1. The method can include culturing the cell under conditions suitable for the production of the appropriate activated sugar-nucleotide. For instance, suitable conditions may include the presence of a sugar in the medium that is transported into the cell and subsequently processed and joined to the appropriate nucleotide.

The method may further include enriching the activated sugar-nucleotide, isolating the activated sugar-nucleotide, or purifying the activated sugar-nucleotide. Methods for enriching, isolating, or purifying activated sugar-nucleotides are known in the art and include, for example, C18 chromatography, anion chromatography, and the use of charcoal and/or DEAE to absorb activated sugar-nucleotide.

In some embodiments, the method can include the use of a genetically engineered cell that exhibits increased phosphorylation of a monosaccharide sugar at the 1 position to produce more of a monosaccharide-1-phosphate when compared to a control cell, and culturing the cell in the presence of the monosaccharide to yield an activated sugar-nucleotide. In one embodiment, the monosaccharide can be galactose, yielding the activated sugar-nucleotide UDP-Gal. In another embodiment, the monosaccharide can be galacturonic acid, yielding the activated sugar-nucleotide UDP-GalA. In yet another embodiment, the monosaccharide can be glucose, yielding the activated sugar-nucleotide UDP-rhamnose.

In some embodiments, the methods may be used to produce labeled activated sugar-nucleotides. In one embodiment, the method can include culturing a genetically engineered cell with a labeled carbon source such as, for example, a C-labeled (e.g., ¹³C-labeled or ¹⁴C-labeled) sugar or sugar precursor. Exemplary carbon sources that may be C-labeled include, for example, glucose, acetate, acetaldehyde 2-oxoglutarate, ethanol, fructose, fumarate, L-glutamine, L-glutamate, D-lactate, L-malate, pyruvate, phosphoenolpyruvate (PEP), galactose, glucuronate, ribulose, and/or succinate. Depending on the location of the label (see, e.g., FIG. 5), the use of labeled glucose in the medium for example, may result in an activated sugar-nucleotide with at least one carbon of the sugar moiety being labeled (C″1, C″2, etc., see e.g., FIG. 17F) and/or at least one carbons of the ribose moiety (C′1, C′2, etc., see, e.g., FIG. 17G) to be labeled.

In another embodiment, the method can include culturing a genetically engineered cell with a source of isotopically labeled nitrogen, e.g., ¹⁵N. Exemplary suitable nitrogen sources include, for example, ammonium chloride and L-glutamine. The use of a labeled nitrogen source may result in an activated sugar-nucleotide with at least one nitrogen being labeled either at the sugar (amino-sugar) or the purine/pyrimidine moiety of a NDP-sugar (see, e.g., FIG. 6).

In another embodiment, the method can include culturing a genetically engineered cell with a source of isotopically labeled hydrogen, e.g., tritium (³H) or deuterium (²H). Exemplary suitable hydrogen sources include, for example, tritium-labeled or deuterium-labeled derivatives of acetate, acetaldehyde 2-oxoglutarate, ethanol, fructose, fumarate, L-glutamine, L-glutamate, D-lactate, L-malate, pyruvate, PEP, galactose, glucose, fucose, glucuronate, ribulose, succinate and/or water (e.g., D₂O). The use of labeled hydrogen can result in at least one hydrogen of the sugar, the ribose, and/or the pyridine/purine moieties of NDP-sugar being labeled.

In another embodiment, the method can include culturing a genetically engineered cell with a source of labeled oxygen, e.g., ¹⁷O or ¹⁸O. Exemplary oxygen sources include, for example, water, arabinose-5P, PEP, glycerol, or molecules that harbor one or more oxygen atoms such as, for example, galactose, galacturonate, glucose, etc. The use of a labeled oxygen source may result in an activated sugar-nucleotide with at least one labeled oxygen in the sugar ring (e.g., C″1-O, C″2-O, etc.) of the NDP-sugar or at least in one or more oxygen of the ribose moiety (e.g., C′2-O, C′3-O, etc.) and/or on the nucleotide ring base (uracil, adenosine, guanosine, cytosine, and thymidine).

In yet another embodiment, the method can include culturing a genetically engineered cell with a source of isotopically labeled phosphorus, e.g., ³²P r ³³P. Exemplary phosphorus sources include, for example, inorganic phosphate (e.g., phosphoric acid, hydrogen phosphates or pyrophosphates) or organic phosphate (e.g., phosphoenolpyruvate (PEP), ATP, DNA, RNA. The use of a labeled phosphorus source may result in an activated sugar-nucleotide or sugar-phosphates with one or more isotopically labeled phosphates attached to the sugar moiety—e.g., glucose-1-P, uridine-P—P-sugar, ribose-5-P.

Activated sugar-nucleotides may be used in research in many areas such as, for example, transport of nucleotide-sugars into organelle, export rate, incorporation of sugars to glycans, glycobiology research, biology of cancer, elucidation of mechanisms that are operative in cell walls, elucidation of composition/structure of polysaccharides by spectrometric methods, and the generation of modified polysaccharides, etc. For example, activated sugar-nucleotides, including those carrying one or more isotopic labels, can serve as standards for methods including, for example, nuclear magnetic resonance, mass spectrometry, radioactive-HPLC, TLC, and/or other chromatography instrumentations. Activated sugar-nucleotides, including those carrying one or more isotopic labels, also can be used to study metabolism, including the study of metabolic diseases. Activated sugar-nucleotides, including those carrying one or more isotopic labels, also can be used in in vivo imaging of cells, organs, tissues, and/or an organism.

In yet another aspect, this disclosure describes a kit for producing an activated sugar-nucleotide. The kit includes at least one genetically engineered cell described herein in a suitable packaging material in an amount sufficient for culturing. Optionally, other reagents such as buffers and solutions needed to practice the invention are also included. Instructions for use of the genetically engineered cell may also be included.

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The term “and/or” means one or all of the listed elements or a combination of any two or more of the listed elements; the terms “comprises” and variations thereof do not have a limiting meaning where these terms appear in the description and claims; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

The present invention is illustrated by the following examples. It is to be understood that the particular examples, materials, amounts, and procedures are to be interpreted broadly in accordance with the scope and spirit of the invention as set forth herein.

### EXAMPLES

### Example 1

[¹³C]Glc and [¹⁵N]NH₄Cl compounds were purchased from Cambridge Isotope Laboratories. Glc is uniformly labeled. M9 and CA media were purchased from DIFCO.

Formation of plasmids harboring UDP-N-acetylglucosamine dehydrogenase and UDP-N-acetylxylose synthase from Bacillus

The coding region encoding the Bacillus cereus UDP-N-acetylglucosamine dehydrogenase, (UGlcNAcDH) was amplified using pET28b:BcUGlcNAcDH as the template along with forward

(5′-TGCTGCCACCGCTGAGCAAATTAATACGACTCACTATAG
GGG-3′ (SEQ ID NO: 1))

and reverse

(5′-CCAAGGGGTTATGCTAGTTATTGCTCAGC-3′
(SEQ ID NO: 2))

primer sets and Phusion Hot Start DNA Polymerase (New England BioLabs; Ipswich, Mass.). The primers were designed with a 15-nucleotide perfect homology (underlined) to the region of pET28 flanking the BlpI site. Following PCR amplification, the DNA was digested with DpnI and purified using a spin column (Qiagen; Valencia, Calif.). The DNA was cloned by in-fusion reaction (according to the manufacturer instructions, Clontech; Mountain View, Calif.) to a BlpI/linearized pET28b-BcUXylNAcS. The resulting plasmid (pET28b:BcUXylNAcS+BcUGlcNAcDH) has two T7 promoters, one for the expression of UDP-N-acetylxylose synthase, UXylNAcS and the other for the expression of UGlcNAcDH.
Formation of Plasmids Harboring Galacturonic Acid Kinase, Galactose Kinase, and UDP-Sugar Pyrophosphorylase from Plants

Plasmids with coding regions encoding Arabidopsis galacturonic acid kinase (GalAK, At3g10700), galactose kinase (GalK, At3g06580) and UDP-sugar pyrophosphorylase (“Sloppy”, At5g52560) were generated as described (Yang et al., J. Biol. Chem. (2009) 284:21526-21535). The NcoI-NotI fragment of Sloppy derived from pET28b:At5g52560#a73f/2#2, was subcloned to the pACy-duet-1 vector to create pACy:At5g52560#6. GalK (pET28a:At3g06580) and Sloppy (pACy:At5g52560) plasmids were co-transformed into the BL21(DE3)-derived E. coli strain (Novagen; Rockport, Mass.) for co-expression and clones were selected on media supplemented with 30 μg/ml chloramphenicol and 50 μg/ml kanamycin. Plasmid harboring GalAK (pET28b:At3g10700) and Sloppy (pACy:At5g52560) were co-transformed into the BL21(DE3)-derived E. coli strain. An empty vector was used as a control.

Formation of plasmids harboring UDP-Glc 4,6-dehydratase and UDP-4keto-6deoxyGlc 3,5-epimerase/4-reductaae

The region encoding the fungal UDP-glucose-4,6-dehydratase (from Botryotinia fuckeliana, GI:335347092), the region encoding the fungal UDP-4-keto-6-deoxyglucose-3,5-epimerase/-4-reductase (from Magnaporthe oryzae, GI:335347090) and the region encoding the plant UDP-sugar pyrophosphorylase (“Sloppy” from Arabidopsis, At5g52560) were amplified individually using Phusion DNA Polymerase. The primers were designed such that all three genes will be incorporated into one plasmid pET28. The resulting plasmid (abbreviated pET28b:Sloppy+Bf4,6dh_Mg3,5Ep/red) has three T7 promoters, one upstream of each coding region. Plasmid harboring the three coding regions was transformed into the BL21(DE3)-derived E. coli strain. In some of these studies the medium was supplemented with [¹³C]Glc for the incorporation of labeled carbon to yield isotope-labeled UDP-Rha.

### Extraction of NDP-Sugars

Bacterial strains (3 ml) harboring the different expression plasmids were grown overnight in LB (10 g Bacto tryptone, 5 g Bacto yeast extract, 10 g NaCl, per liter) or M9/CA (sodium-phosphate dibasic 6.78 g, potassium phosphate monobasic 3 g, NaCl 0.5 g, ammonium chloride 1 g, and casamino acid 8 g per liter) medium supplemented with chloramphenicol (30 μg/ml) and kanamycin (50 μg/ml) at 37° C. at 250 rpm. Portions of the culture media were used to inoculate fresh media (5 ml) and allowed to grow to an OD_{600 nm} of 0.4 and 0.6. The medium was then supplemented with an appropriate carbon source (sugar or ¹³C-labeled sugar at 0.2% w/v) or nitrogen source ([¹⁵N]NH₄Cl at 0.2% w/v). Isopropyl β-D-thiogalactoside (0.5 mM, IPTG) was added and the cells were allowed to grow for up to four hours at the indicated temperature. A portion (3 ml) of the culture was removed and centrifuged (18,000×g, 1 min, 22° C.). The cell pellet was washed twice with four volumes of 10 mM Na-phosphate pH 7.5, 150 mM NaCl (PBS) and then suspended in 75 mM NaF. Ten volumes of cold chloroform:methanol (1:1, v/v) was added, and the sample was mixed for 30 minutes on ice. The suspension was centrifuged (18,000×g, 4 minutes, 22° C.) and the upper aqueous phase was collected and re-centrifuged. Portions of this aqueous phase were analyzed by high-performance anion-exchange chromatography, liquid chromatography, electrospray-ionization mass spectrometry (LC-ESI-MS/MS) and ¹H-NMR spectroscopy.

### Analysis of NDP-Sugars

The aqueous extracts of the E. coli cells were separated on a Q15 anion-exchange column (1 mm id×250 mm, Amersham (now GE Healthcare); Niskayuna, N.Y.) using an Agilent Series 1100 HPLC system equipped with an autosampler, diode-array detector and ChemStation software Ver. B.04.02. Samples (30 μl) were injected and the column was washed at 0.2 ml/min for three minutes with 20 mM ammonium formate, pH 8.4. Nucleotides were then eluted with a linear gradient from 20 mM to 500 mM ammonium formate over 23 minutes. Nucleotides were detected by their A_{261 nm} and quantified using calibration curves generated from standard UDP-sugars. The peaks corresponding to UDP-GlcNAcA (Rt 23.2 min), UDP-XylNAc (Rt 16.0 minutes), UDP-Gal (Rt 16.8 minutes), and UDP-GalA (Rt 23.8 minutes) were collected, lyophilized and analyzed by NMR spectroscopy and by ESI-MS/MS. Column fractions (0.4 ml) were collected and either lyophilized and reconstituted in D₂O (150 μl) for ¹H-NMR analysis, or a portion was analyzed by LC-ESI-MS/MS.

For ESI-MS analysis, each column fraction (47.5 μl of the 0.4 ml) or standard nucleotide-sugar (1 μM to 100 μM in 47.5 μl) was mixed with 2.5 μl 0.4 M triethylammonium acetate (TEAA), pH 7 (Sigma), and 50 μl acetonitrile (AcN). Samples were analyzed using a linear ion trap mass spectrometer (LTQ-XL, ThermoFisher) with direct infusion at a rate of 3-10 μl/minute using a Harvard syringe pump. Samples (100 μl) were also introduced via the HPLC autosampler at 25 μl/minute with 10 mM TEAA, pH 7 in 50% can (Turnock and Ferguson, Eukaryot Cell (2007) 6:1450-1463). Negative ion spectra were recorded over the mass range m/z=100-1,000. Prominent ions in the mass spectrum were selected and subjected to collision-induced dissociation (CID) with a helium collision gas pressure of 3×10⁻³ torr and collision voltage set to 25 to give an MS/MS product ion spectrum. UDP-Glc (exact theoretical mass of 566.047) gave a [M-H]⁻ ion at m/z 565.08 and when fragmented produced a major product ion at m/z 323.00 corresponding to a [UMP-H]⁻ ion (UMP Calc. theoretical mass of 324.036). The MS assignments of other NDP-sugar are summarized in Table 2.

TABLE 2
MS/MS fragmentation of the sugar nucleotides

| | CID -fragmentation | Major MS/MS |
|---|---|---|
| Sugar Nucleotide | of parent ion (m/z) | ion product (m/z) |
| UDP-Glc | 565→323 | [UMP-H]⁻ |
| UDP-Gal | 565→323 | [UMP-H]⁻ |
| UDP-GalA | 580→403 | [UDP-H]⁻ |
| UDP-XylNAc | 576→385 and 403 | [UDP-H-H₂O]⁻and [UDP-H]⁻ |
| UDP-GlcNAcA | 620→403 | [UDP-H]⁻ |

Mass spectrometry was performed in the negative ion mode.

Analyses of ³C- or ¹⁵N-Labeled NDP-Sugars by NMR Spectroscopy

Overnight cultures of E. coli were diluted into M9/CA media and grown to OD_{600 nm}=0.6. The culture (5 ml) was induced by IPTG (0.5 mM); [¹³C]Glc or [¹⁵N]NH₄Cl were added (0.2%); and culture were grown up to four hours. After NDP-sugar extraction, sample was chromatographed and HPLC-column fractions (0.4 ml) were collected, lyophilized, and then dissolved in D₂O (150 μl) for NMR spectroscopic analysis. 1D ¹H and 2D ¹³C heteronuclear single-quantum coherence (HSQC) or ¹⁵N heteronuclear multiple bond correlated (HMBC) spectra of the HPLC-purified UDP-GlcNAcA or UDP-XylNAc (lyophilized, and resuspended in 150 μl D₂O) were collected at 25° C. using a Varian/Agilent DirectDrive™ 600 MHz and 900 MHz spectrometers. The HSQC spectra were collected with a carbon spectral width of 170 ppm, centered at 75.1 ppm, and with 128 points of 200 transients each. The HMBC spectra were collected with a nitrogen width of 65.8 ppm, centered at 139.6 ppm, and with 64 points of 400 transients each.

### Example 2

### Synthesis of Short-Lived Nucleotide Sugars Engineered Biological

Certain NDP-sugars are not stable and undergo spontaneous degradation. For example, CMP-KDO, is hydrolyzed to CMP and KDO in less than one hour (Lin et al., Biochemistry (1997) 36: 780-785). UDP-Apiose is degraded to UMP and 1,2-P cyclic apiose (Guyett et al., Carbohydr Res (2009) 344: 1072-1078). The use of such relatively short-lived molecule in research is a limiting factor. We tested the engineered biological system to determine whether it may be used to generate short-lived NDP-sugars in amounts reasonable for work.

A bacterial strain harboring CMP-KDO synthase (E. coli KDSB (Goldman et al., J Bacteriol (1985) 163: 256-261) or plant KDS (Royo et al., J Biol Chem (2009) 275: 24993-24999) were grown overnight in LB media supplemented with appropriate antibiotics (30 μg/ml chloramphenicol, 50 μg/ml kanamycin) at 37° C. at 250 rpm. A portion of the culture was used to inoculate fresh media and allowed to grow to OD₆₀₀=0.6 to 0.8. Fresh media was supplemented with KDO at final concentration to 0.2% and isopropyl β-D-thiogalactoside (0.5 mM, IPTG) was added. After further growth for four hours at 37° C., 3 ml of culture was removed, centrifuged (14,000 rpm, 1 minute, 22° C.), and the cell pellet was washed twice with 4 volumes of PBS (10 mM Na-phosphate pH 7.5, 150 mM NaCl). The cells were suspended with NaF (75 mM) and ten volumes of cold organic solution (chloroform:methanol, 1:1 v/v) was added. The sample was mixed for 10 minutes on ice, centrifuged (14,000 rpm, 4 minutes, 22° C.) and the upper phase was collected and re-centrifuge. An aliquot was analyzed by HPLC-UV and ESI-MS/MS.

Our engineered E. coli was able to produce CMP-KDO in the cell, which was detectable by LC-MS. (FIG. 9).

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A genetically engineered cell exhibiting an increase in synthesis of an activated sugar-nucleotide compared to a wild type control.

2. The genetically engineered cell of claim 1 wherein the activated sugar-nucleotide is an activated uridine diphosphate sugar nucleotide.

3. The genetically engineered cell of claim 2 wherein the activated uridine diphosphate sugar nucleotide is UDP-Gal, UDP-GalA, UDP-Rha, UDP-GlcNAcA, or UDP-XylNAc.

4. The genetically engineered cell of claim 1 wherein the activated sugar-nucleotide is an activated cysteine monophosphate sugar nucleotide.

5. The genetically engineered cell of claim 4 wherein the activated cysteine monophosphate sugar nucleotide is CMP-KDO or CMP-KDO-N₃.

6. The genetically engineered cell of claim 1 wherein the activated sugar-nucleotide is an activated guanosine diphosphate sugar nucleotide.

7. The genetically engineered cell of claim 6 wherein the activated guanosine diphosphate sugar nucleotide is GDP-Man or GDP-L-Gal.

8. The genetically engineered cell of claim 1 wherein the activated sugar-nucleotide comprises an isotopic label.

9-10. (canceled)

11. The genetically engineered cell of claim 1 wherein the cell comprises a prokaryote.

12. (canceled)

13. The genetically engineered cell of claim 1 wherein the cell comprises a eukaryote.

14. The genetically engineered cell of claim 1 wherein activated sugar-nucleotide comprises an activated sugar-nucleotide that is not natively synthesized by the wild type control.

15. (canceled)

16. A method for making an activated sugar-nucleotide, the method comprising:
providing a genetically engineered cell exhibiting increased phosphorylation of a monosaccharide sugar at the 1 position to produce a monosaccharide-1-phosphate compared to a wild-type bacterial cell; and
culturing the cell in the presence of the monosaccharide to yield the activated sugar-nucleotide.

17. The method of claim 16 wherein the monosaccharide sugar is galactose and the activated sugar-nucleotide is UDP-galactose.

18. The method of claim 16 wherein the monosaccharide sugar is galacturonic acid and the activated sugar-nucleotide is UDP-galacturonic acid.

19-24. (canceled)

25. A method for making an activated sugar-nucleotide, the method comprising:
providing a genetically engineered cell exhibiting increased production of an activated sugar-nucleotide compared to a wild-type cell; and
culturing the cell under conditions suitable for production of the activated sugar-nucleotide.

26. (canceled)

27. The method of claim 25 wherein the activated sugar-nucleotide is a UDP sugar-nucleotide, a CMP sugar-nucleotide, or a GDP sugar-nucleotide.

28-33. (canceled)

34. An isotopically labeled activated sugar-nucleotide.

35. (canceled)

36. The isotopically labeled activated sugar-nucleotide of claim 34 wherein the isotopic label comprises ²H, ¹³C, ¹⁴C, ¹⁵N, ¹⁷O, ¹⁸O, ³²P, or ³³P.

37-38. (canceled)

39. A method comprising:
performing an assay using the isotopically labeled activated sugar-nucleotide of claim 34.

40-45. (canceled)

46. The method of claim 39 wherein:
the assay comprises providing the isotopically labeled activated sugar-nucleotide to a cell and detecting the isotopic label; and
tracking movement of the isotopic label.

47. The method of claim 46 further comprising imaging the isotopic label.
